(19) United States
(12) Patent Application Publication
(10) Pub. No.: US 2026/0078162 A1
(43) Pub. Date: Mar. 19, 2026

(54) REVERSIBLE CONTROL OF CHIMERIC ANTIGEN RECEPTOR T CELLS

(71) Applicant: Dana-Farber Cancer Institute, Inc., Boston, MA (US)

(72) Inventors: Xin Zhou, Boston, MA (US); DingPeng Zhang, Boston, MA (US)

(21) Appl. No.: 19/113,763

(22) PCT Filed: Sep. 20, 2023
(86) PCT No.: PCT/US2023/074692

§ 371 Date: Mar. 20, 2025

Related U.S. Application Data
(60) Provisional application No. 63/376,389, filed on Sep. 20, 2022.
(60) Provisional application No. 63/462,828, filed on Apr. 28, 2023.

(51) Int. Cl. C07K 14/705; A61K 38/00

(52) U.S. Cl. CPC C07K 14/70503; C07K 14/70578; A61K 38/00; C07K 2319/30

(57) ABSTRACT

Disclosed are receptor traps that regulate immune cells. The receptor traps can have an ectodomain from a tumor cell that can bind to a chimeric antigen receptor (CAR) on a CAR-T cell that reversibly inhibits activation of the CAR-T cell.

This application claims priority to U.S. Provisional Application No. 63/376,389, filed on Sep. 20, 2022, and U.S. Provisional Application No. 63/462,828, filed on Apr. 28, 2023, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

Aspects of the invention are drawn to compositions and methods for modulating molecules on cell surfaces, including chimeric antigen receptors (CAR) on the surface of CAR-T cells, to reversibly control CAR receptors and receptors on cancer cells to inhibit cancer cell signaling and growth.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### BACKGROUND OF THE INVENTION

Chimeric antigen receptor (CAR) T cells have emerged as promising therapies for patients with hematologic malignancies. However, widespread use of CAR-based immunotherapies has been limited by potentially life-threatening toxicities, and the therapeutic effect is limited by attenuated persistence. Cytokine-release syndrome (CRS) and immune effector cell-associated neurotoxicity syndrome (ICANS) are the two most-common toxicities observed after CAR-T cell therapy. Further, attenuated persistence is often due to immune cell exhaustion, in which continued signaling through the CAR ultimately leads to low or no efficacy and therapeutic failure or relapse. Thus, a desirable CAR-based immunotherapy would include the ability to limit toxicity due to unwanted downstream signaling effects and to control or limit CAR activity to avoid exhaustion.

### SUMMARY OF THE INVENTION

Disclosed here are new reagents and methods for regulating molecules on the surface of cells. In some embodiments, the reagents and methods are used to control the levels of CAR molecules on CAR-T cells. In some embodiments, the reagents and methods are used to reversibly control (e.g., inhibit) CAR-T cell activation. In some embodiments, CAR-T cell activation is controlled in vivo. In some embodiments, the reagents and methods are used to control or inhibit cancer cell growth or modulate immune responses.

In some embodiments, disclosed are antigen or receptor traps as described herein. In some embodiments, the antigen/receptor traps have an ectodomain or ectodomain means to which a chimeric antigen receptor (CAR) can bind (e.g., an extracellular region of a cellular membrane protein that can be bound by a CAR on a CAR-T immune cell; e.g., an ectodomain-based ligand trap for an immune cell). In some embodiments, an ectodomain means can be bound by the CAR-T cell, in substantially the same way that an ectodomain can be bound by the CAR, but the ectodomain means can be derived from, a variant of, and the like, of an ectodomain.

In some embodiments, the ectodomain means can be fused to a dimerization means. The dimerization means can cause two receptor traps to form a dimer. Dimerization can increase valency of the receptor trap. In some embodiments, the dimerization means can be an Fc portion of an antibody. In some embodiments, binding of the trap molecule by the CAR can block CAR-T cells (e.g., inhibit binding of the CAR to a molecule that can activate a CAR-T cell; inhibit CAR-T cell activation). In some embodiments, the blockage or inhibition can be reversible. In some embodiments, binding of the trap molecule by the CAR does not activate or does not substantially activate the CAR-T cell. In some embodiments, the antigen/receptor traps can be called CAR-Trap molecules. In some embodiments, then CAR-Trap molecules are multivalent.

Disclosed are nucleic acids encoding these molecules, vectors that contain the nucleic acids, and cells that contain the vectors and/or express the receptor trap as disclosed herein.

In some embodiments, disclosed are methods of administering the antigen/receptor traps to a subject.

### BRIEF DESCRIPTION OF THE DRAWINGS

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 is a schematic illustrating CAR-T cell therapy in a patient.

FIG. 2 illustrates an example approach for treating toxicities observed after CAR-T-cell therapy using immunosuppressive agents.

FIG. 3 illustrates an example approach for treating toxicities observed after CAR-T-cell therapy using suicide genes or elimination markers.

FIG. 4 illustrates an example approach for treating toxicities observed after CAR-T-cell therapy using reversible genetic switches.

FIG. 5 illustrates reversible CAR-T regulatory mechanisms as strategies to enhance CAR-T cell efficacy.

FIG. 6 illustrates an example approach for controlling CAR-T cell activation using receptor traps, as disclosed herein.

FIG. 7 illustrates an example of expression of CD19 receptor trap monomers and dimers.

FIG. 8 illustrates an example of grafting a CD19 extracellular domain to human IgG1 FC and expression of the molecules.

FIG. 9 illustrates an example of an anti-CD19 Jurkat NFAT GFP/CD19-K562 co-culture assay.

FIG. 10 illustrates results of a CD19 receptor trap monomer blocking anti-CD19 Jurkat CAR activation in a dose-dependent manner.

FIG. 11 illustrates results of a CD19-Fc dimer receptor trap molecule that is more effective than a monomer in blocking anti-CD19 Jurkat CAR activation.

FIG. 12 illustrates results of improved expression yield and reduced aggregation of engineered CD19 receptor trap variants.

FIG. 13 illustrates an example of grafting improved CD19 extracellular domains to human IgG1 FC and expression of the molecules.

FIG. 14 illustrates results of engineered CD19 receptor trap variants blocking anti-CD19 Jurkat CAR activation with improved IC₅₀'s.

FIG. 15 illustrates results showing that receptor trap inhibition of Jurkat cell activation is reversible.

FIG. 16 illustrates additional results showing that receptor trap inhibition of Jurkat cell activation is reversible.

FIG. 17 illustrates additional results showing that receptor trap inhibition of Jurkat cell activation is reversible.

FIG. 18 illustrates results of antigen/receptor trap inhibition of Jurkat cell activation with the presence of K562, and antigen/receptor traps minimally influencing Jurkat CAR-T cells in the absence of activating tumor cells.

FIG. 19 illustrates additional results of antigen/receptor traps minimally influencing Jurkat CAR-T cells in the absence of activating tumor cells.

FIG. 20 illustrates additional results of antigen/receptor traps minimally influencing Jurkat CAR-T cells in the absence of activating tumor cells.

FIG. 21 illustrates additional results of antigen/receptor traps minimally influencing Jurkat CAR-T cells in the absence of activating tumor cells.

FIG. 22 illustrates additional results of antigen/receptor traps minimally influencing Jurkat CAR-T cells in the absence of activating tumor cells.

FIG. 23 illustrates expression of antigen/receptor traps containing HER2 extracellular domain, an epitope from the HER2 protein to which the Herceptin antibody binds (Herceptin BD-FC), or an extracellular domain of BCMA.

FIG. 24 illustrates results of expression of CARs specific for CD19, HER2 or BCMA in primary T cells.

FIG. 25 illustrates schematic diagrams of retroviral vectors used to express the various CARs.

FIG. 26A, FIG. 26B and FIG. 26C illustrate activity of antigen/receptor traps shown in FIG. 32 for BMCA, HER2 and HER2, respectively. Each panel shows results without target cells (left) and with target cells (right).

FIG. 27A, FIG. 27B and FIG. 27C illustrate a schematic diagram of a study used to detect effects of CARTrap 9, specific for CD19 (A), on the effects of cytokine production (B) and tumor cell killing (C) of primary cells. The data show that the CARTrap inhibited/stopped interferon gamma production (B). The data show that the CARTrap stopped tumor killing and, by removing the CARTrap, tumor killing resumed.

FIG. 28A-E. Characterization of CD19 Ectodomain Variants for Inhibiting CAR-T Cell Activities. (A) Depicts a schematic representation of CD19 wt, NT.1, 19.1, C6.2, and CT.2 fused with IgG1 Fc to form CAR-Traps. (B) Shows dose-dependent inhibition of CAR-Jurkat cell activation by K562 cells with various CD19 CAR-Trap molecules and corresponding IC50 values. CAR-Jurkat cell activation levels were measured using NFAT-GFP via flow cytometry. (C) Shows a SDS-PAGE gel characterization of the five CAR-Trap proteins. (D) Shows a comparison of protein expression levels for CD19 ectodomain variants CAR-Traps with the CD19 wt CAR-Trap. (E) Shows structural illustration of the CD19/CAR scFv complex, indicating that mutations in CD19 NT.1 are primarily located far from the interaction interface. The model was constructed using PyMOL based on PDB 7URV.

FIG. 29A-E. Characterization of Multivalent CD19 Ectodomain Fusions for CAR-T Cell Activity Inhibition. (A) Shows a schematic representation of CD19 NT.1 monomer, dimer, and tetramer. (B) Shows SDS-PAGE gel confirmation of monomer, dimer, and tetramer fusion protein formation. (C) Shows dose-dependent inhibition of CAR-Jurkat cell activation by K562 cells with varying CAR-Trap molecules, along with corresponding IC50 values. CAR-Jurkat cell activation levels were quantified using NFAT-GFP through flow cytometry. (D) Depicts illustrations comparing monomeric versus dimeric CAR-Traps in competition with tumor surface antigens for CAR binding on the T cell membrane. Given the concurrent engagement of multiple CARs and tumor antigens at the cancer cell-T cell interface, a dimeric CAR-Trap would exhibit superior capability in disrupting this interaction. (E) Shows reversibility of CAR-Jurkat activation with CAR-Trap.

FIG. 30A-D. (A) Depicts a schematic representation of CAR density impact on the baseline, ligand-independent tonic signaling as well as the response to both CAR-Traps and tumor cells. (B) Depicts strength of various CAR-T cell signals. (C) Different expression groups for Jurkat cells expressing an EF1a-CAR construct either untreated or exposed to varying concentrations of CAR-Traps, or tumor cells at a 1:1 effector:T cell ratio. (D) CD69 levels for the various groups included in (C).

FIG. 31A-D. CAR-Trap reversibly regulates primary CAR-T cells. (A) Schematic of the setup of a primary CAR-T cell co-culture assay. Secreted IFN-γ levels are measured to determine CAR-T cell activation levels in the presence if CD19+ A375 cells and CAR-Traps; live cell fluorescence microscopy is used to determine the antitumor effects. (B) Measurement of human primary CAR-T cell IFN-γ release in the co-culture assay described in (a) with an IFNγ split-luciferase assay (Promega). Data are representative of 2 independent experiments. (C) Fluorescence microscopy of mCherry-labeled A375 cells showing CAR-T cell-mediated A375 killing reversibly controlled with CAR-Trap. (D) Overlay of bright field and mCherry channel images showing CAR-T cell-mediate A375 killing activity was resumed after CAR-Trap washout over time.

32A-C. Engineering of BCMA CAR-Trap. (A) Structure of a BCMA ectodomain, derived from PDB 4ZFO. (B) Schematic representation of a BCMA CAR-Trap and its corresponding SDS-PAGE validation. (C) Dose-dependent inhibition of anti-BCMA CAR-Jurkat cell activation by H929 cells with the BCMA CAR-Trap, along with the corresponding IC50 value.

### DETAILED DESCRIPTION OF THE INVENTION

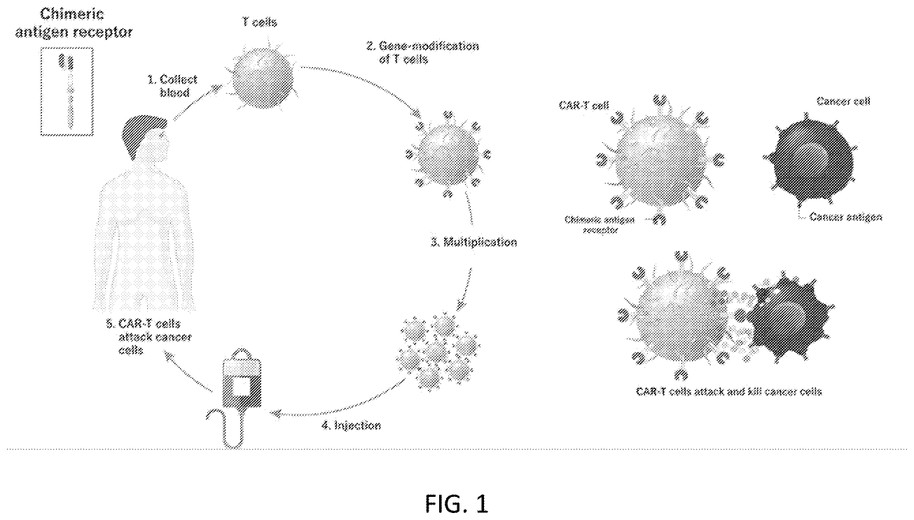

Chimeric antigen receptor (CAR) T cells have emerged as a promising therapy for patients with hematologic malignancies (FIG. 1). In some cases, however, CAR-T therapies can cause side effects in patients who receive these cells, including cytokine-release syndrome (CRS) and immune effector cell-associated neurotoxicity syndrome (ICANS).

Several regulatory mechanisms to control CAR-T cells in vivo have been developed to address these adverse events. However, these strategies have not satisfactorily met current needs since toxicities and fatalities continue to be reported in CAR-T clinical trials.

Herein are disclosed new, modular, and reversible strategies for modulating CAR-T cell activities. Generally, the methods do not require additional genetic engineering of CAR-T cells. In some embodiments, these strategies can modulate CAR-T toxicities. In some embodiments, these strategies can increase efficacy of CAR-T cell therapy. In some embodiments, these strategies are based on reversible blockade or internalization of CAR receptors.

Disclosed herein are recombinant protein switches based on the ectodomains of cell surface molecules to which chimeric antigen receptors (CARs) can bind. These protein switches can be called “CAR-Traps.” In some embodiments, the ectodomains can be tumor-cell surface antigens. CAR-Traps can reversibly and quantitatively “tune” CAR-T cell activity.

In some embodiments, antigen traps, including receptor traps, can include antigens or ligands to which CAR receptors can bind. The receptor traps can function as CAR OFF-switches by binding to the CAR receptor and blocking its interaction with the antigen or ligand that would normally be found on tumor cells to which the CAR-T cells bind, triggering CAR-T cell activation.

For example, the antigen/receptor trap molecule can be a molecule to which a CAR of a CAR-T cell specifically binds. In some embodiments, binding of the antigen/receptor trap molecule by the CAR inhibits the CAR from binding the same or similar molecule on the surface of tumor cells. In some embodiments, binding of the antigen/receptor trap by the CAR prevents or decreases activation of the CAR-T cell. In some embodiments, binding of the antigen/receptor trap by the CAR can activate the CAR-T cell.

In some embodiments, the receptor traps do not require engineering of the CAR-T receptor or CAR-T cells and can be applied to CAR-T therapies that are already approved or in clinical development.

In other embodiments, the receptor traps can be reversible. Reversibility can provide for fine tuning of CAR-T cell activities, for example for toxicity management and/or to rejuvenate the cells for continued treatment.

In some embodiments, the receptor traps can be tailored to CAR-T cells that target different tumor antigens, for example, by replacing the components used in the designs (i.e., the traps can be modular). These antigen traps can be designed by engineering ectodomains of tumor surface antigens. The antigen traps can interrupt the interaction between CAR-T cells and tumor cells.

Also disclosed are approaches for enhancing CAR-T efficacy. Temporal “rest” of CAR-T cells can reverse CAR-T exhaustion. In some embodiments, by alternating CAR-T cells between an “active” and a “resting” state, the disclosed reversible CAR modulators can increase efficacy of CAR-T cells.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

### CAR-T Cells, Toxicities and Controlling Toxicities

Chimeric antigen receptor (CAR) T cells have emerged as a promising treatment for patients with advanced B-cell cancers (FIG. 1). However, widespread application of the therapy can be limited by potentially life-threatening toxicities due to a lack of control of the transfused CAR-T cells. Toxicities are an obstacle for the development of CAR-T therapy for both blood cancers and solid tumors. Reported deaths from CAR-T therapies have recently been discussed in the literature (Neelapu, Sattva S., et al. “Toxicity management after chimeric antigen receptor T cell therapy: one size does not fit ‘ALL’.” Nature reviews Clinical oncology 15.4 (2018): 218-218).

Cytokine-release syndrome (CRS) and immune effector cell-associated neurotoxicity syndrome (ICANS) are the two most-common toxicities observed after CAR-T-cell therapy.

CRS can be characterized by high fever, hypoxia, hypotension, or multiorgan toxicity; it develops in 37%-93% patients with lymphoma and 77%-93% with leukemia. ICANS is characterized by confusion, delirium, seizures, or cerebral oedema; it develops in 23%-67% patients with lymphoma and 40%-62% with leukemia. Severe CRS and ICANS require monitoring and treatment in the intensive-care setting, and multiple fatalities have been reported due to unmanageable CRS or ICANS toxicities.

Three types of treatments for these toxicities are currently in use.

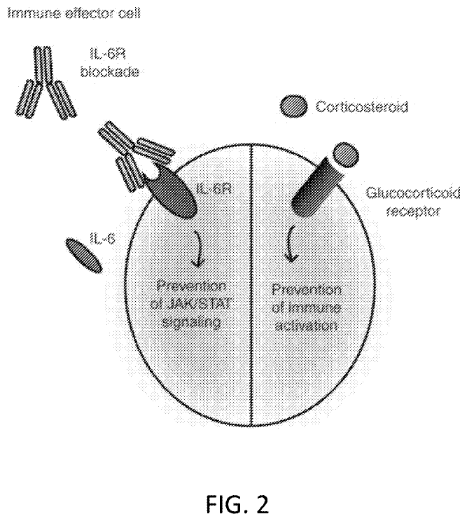

In some instances, patients are treated with immunosuppressive agents (FIG. 2), including systemic corticosteroids, IL-6 receptor antibody (e.g., tocilizumab), lymphocytotoxic anti-CD52 antibody (e.g., alemtuzumab), tyrosine kinase inhibitors (e.g., dasatinib) (LCK inhibitors do not inhibit already activated T cells) and the like. There are limitations to these treatments, however. For example, treatment with high-dose steroids can limit the time span over which CAR-T cells are functional and can induce hematological aplasia and toxicity. Anti-IL6 receptor antibody has a variety of biological activities and can non-specifically inhibit the immune system (Bonifant, Challice L., et al. “Toxicity and management in CAR T-cell therapy.” Molecular Therapy-Oncolytics 3 (2016): 16011).

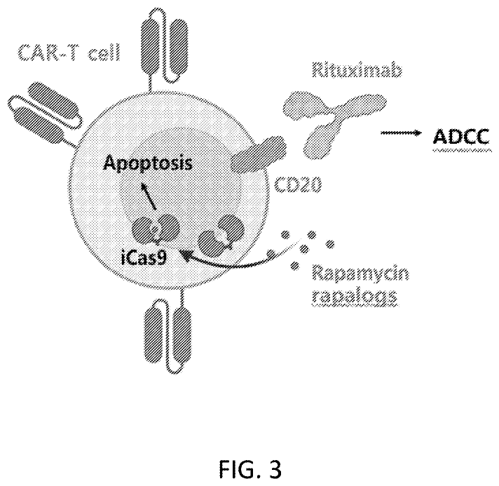

In some instances, patients are treated with suicide genes or elimination markers (FIG. 3), including iCasp9, anti-CD20 (e.g., rituximab), anti-EGFR (e.g., cetuximab) and the like. There are limitations to these treatments, however. For example, these treatments can irreversibly and/or permanently eliminate CAR-T cells from the body (Brandt, Lærke J B, et al. “Emerging approaches for regulation and control of CAR T cells: a mini review.” Frontiers in Immunology 11 (2020): 326).

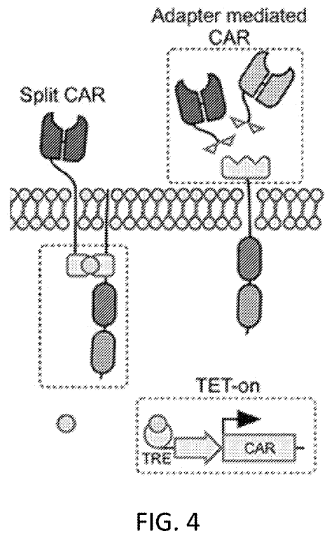

In some instances, CAR-T cells that have switchable CAR receptors can be used in patients (FIG. 4), including split-CAR, SMaSh-CAR, CAR PROTAC and the like. There are limitations to these treatments, however. For example, these treatments can compromise CAR-T activity, the switches can be leaky, and the switches can be immunogenic (Labanieh, Louai, et al. “Enhanced safety and efficacy of protease-regulated CAR-T cell receptors.” Cell 185.10 (2022): 1745-1763).

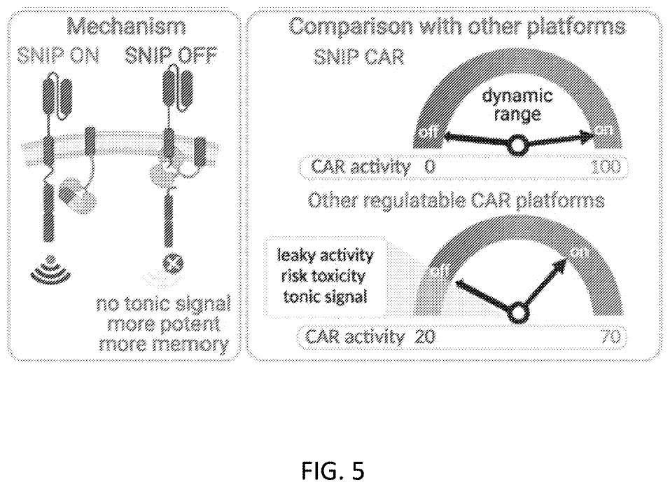

It is known, however, that reversible CAR-T regulatory mechanisms can be used to enhance CAR-T efficacy (FIG. 5). Constitutive CAR-T cells can manifest increased levels of exhaustion-associated proteins. In some embodiments, however, transient “rest” can reverse the exhaustion phenotype. In some embodiments, regulated CAR can be reversibly turned off and on to switch the CAR-T cells between an “Off” and “On” state (Weber, Evan W., et al. “Transient rest restores functionality in exhausted CAR-T cells through epigenetic remodeling.” Science 372.6537 (2021): eaba1786; Labanieh, Louai, et al. “Enhanced safety and efficacy of protease-regulated CAR-T cell receptors.” Cell 185.10 (2022): 1745-1763). This can be a method to treat CAR-T cell exhaustion.

In some embodiments of the invention disclosed herein, antigen/receptor traps are used to regulate CAR-T cells. In some embodiments, these receptor traps can reversibly modulate CAR-T cells.

### Antigen or Receptor Traps

In some embodiments, strategies disclosed herein for regulating a molecule (e.g., the amount of the molecule) on a cell surface and/or for regulating activity of a molecule on a cell surface can use an antigen trap or receptor trap approach. For example, this strategy can involve a cell-surface molecule (e.g., a protein) binding a “trap” molecule. In some embodiments, the trap molecule can be ectodomain or ectodomain means that a chimeric antigen receptor (CAR) on the surface of a CAR-T cell can bind. In some embodiments, the ectodomain means can be bound by the CAR-T cell in substantially the same way that an ectodomain can be bound by the CAR, but the ectodomain means can be derived from, a variant of, and the like, of an ectodomain.

In embodiments, binding of a trap molecule can affect the ability of common ligands of the cell-surface molecule to bind to the cell-surface molecule (e.g., CAR) and/or regulate that molecule. In embodiments, a trap molecule can bind to the cell-surface molecule such that binding of a regulator protein to the cell-surface molecule (e.g., an activator or repressor of the cell-surface molecule; e.g., an epitope to which the CAR can bind) is inhibited. In embodiments, binding of trap molecules themselves to the cell-surface molecule minimally affects regulation of the cell-surface protein (e.g., trap binding does not affect or minimally affects upregulation and/or downregulation of the cell-surface or membrane molecule/protein). In some embodiments, the trap molecules are not associated with a cell (e.g., free in solution). In some embodiments, the trap molecules can also have an Fc region of an antibody fused to the trap molecule, or an ectodomain thereof.

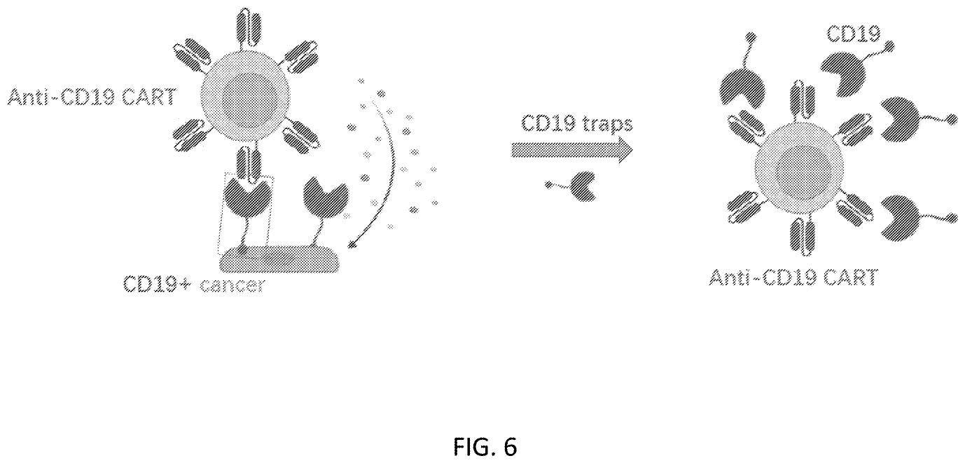

In some embodiments, the cell-surface molecule or protein can be a CAR molecule. In some embodiments, the CAR molecule can be on a CAR-T cell. In some embodiments, a strategy for regulating CAR-T activities includes blocking of CAR receptors with an antigen/receptor trap molecule(s). In some embodiments, blocking of CAR receptors by the trap is reversible. In some embodiments, the receptor traps can function as a CAR OFF-switch by binding to a CAR receptor on T cells and blocking its interaction with the molecule (e.g., the molecule on a cell) to which the CAR receptor normally binds (FIG. 6). In some embodiments, the receptor traps can inhibit activation of the CAR-T cell that binds it. In some embodiments, antigen-receptor trap molecules that are specific for CARs can be called CAR-Trap molecules.

In some embodiments, a receptor trap can be any antigen or epitope to which a chimeric antigen receptor (CAR) specifically binds, with the proviso that: i) the receptor trap molecule blocks binding of at least some “normal” antigens/epitopes that the CAR is designed to bind; and/or ii) does not activate or repress, or minimally activates or represses, the CAR-T cell, by binding to the CAR (e.g., the trap molecule does not have the same regulatory effect on the CAR as does the antigen/epitope on a cell that the CAR is designed to bind). In some embodiments, the receptor trap can reversibly regulate a CAR-T cell.

In embodiments, the receptor trap can be a modification of the antigen/epitope the CAR is designed to bind (e.g., an ectodomain means). In some embodiments, the trap molecule can be similar to the antigen on a cell surface that the CAR is designed to bind. In some embodiments, the ectodomain means can have at least a 10-, 20-, 30-, 40-, or 50-fold lower IC50 for binding to a CAR than does a wild-type ectodomain that the same CAR can bind. In some embodiments, the trap molecule is not associated with a cell.

In some embodiments, the antigens or epitopes to which CARs bind, and which are used in antigen/receptor traps, are those that are specific to or substantially exclusive to tumor or cancer cells (e.g., tumor-specific antigens, tumor-associated antigens and the like). That is, at least in some embodiments, the antigens or epitopes that are used in antigen/receptor traps are not found, or are minimally present, on non-tumor or non-cancer cells.

In some embodiments, the trap molecules can be from tumor-specific antigens (TSAs) or tumor-associated antigens (TAAs).

In embodiments, the trap molecules can be from blood (hematological) cancer cells or cells from solid tumors. In embodiments, the target antigens or epitopes can be from B-cell acute lymphoblastic leukemia (ALL), B-cell non-Hodgkin lymphoma (NHL), follicular lymphoma, mantle cell lymphoma (MCL), multiple myeloma and the like. In embodiments, the target antigens or epitopes can be from brain tumors, breast tumors, kidney tumors and the like.

In embodiments, the target antigens or epitopes can be from hepatocellular carcinoma, GPC3 positive hepatocellular carcinoma, hepatic carcinoma, lung cancer, advanced lung cancer, advanced solid tumors, colon cancer, colorectal cancer, EGFR-positive colorectal cancer, esophageal carcinoma, pancreatic cancer, prostate cancer, gastric cancer, sarcoma, osteoid sarcoma, Ewing sarcoma, breast cancer, ovarian cancer, glioma, cervical cancer, squamous cell lung cancer, liver metastases, liver neoplasms, stomach neoplasms, advanced EGFR-positive solid tumors and the like.

In some embodiments, the antigen/receptor trap molecules are based on ectodomains or ectodomain means from CD19 or B-cell maturation antigen (BCMA).

In some embodiments, the engineered ectodomains can be multivalent (e.g., multimers).

In some embodiments, density of CAR molecules on CAR-T cells can affect CAR-T cell response to CAR-Traps and tumor cells.

In embodiments, the target antigen or epitopes used in antigen/receptor traps can include CD19, B cell maturation antigen (BCMA), human epidermal growth factor 2 (HER2) and the like.

In some embodiments, a CD19 ectodomain or ectodomain means can be (SEQ ID NO: 26):

PEEPLVVKVEEGDNAVLQCLKGTSDGPTQQLTWSRESPLKPFLKLSLGL
PGLGIHMRPLAIWLFIFNVSQQMGGFYLCQPGPPSEKAWQPGWTVNVEG
SGELFRWNVSDLGGLGCGLKNRSSEGPSSPSGKLMSPKLYVWAKDRPEI
WEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRG
NLTMSFHLEITARPVLWHWLLRTGGWK

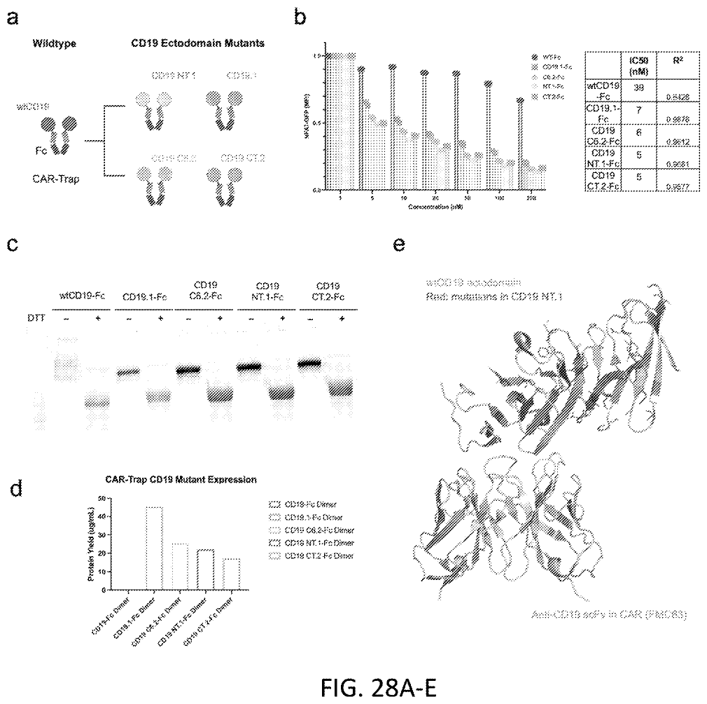

In some embodiments, the CD19 ectodomain can be a variant with a lower IC50 than the wild-type molecule. In some embodiments, the CD19 ectodomain can be a 19.1, C6.2, NT.1, CT.2 variant of CD19 (FIG. 28A).

In some embodiments, a BCMA ectodomain or ectodomain means can be (SEQ ID NO: 27):

LQMAGQCSQNEYFDSLLHACIPCQLRCSSNTPPLTCQRYCNASVTNSVK
GTNA

In embodiments, the target antigen or epitope used in antigen/receptor traps can include molecules from hematological cancers. In embodiments, the target antigen or epitope used in antigen/receptor traps can include CD123, CD138, CD20, CD22, CD38, CD5, Ig κ chain, LeY, NKG2D ligand, ROR1, WT1 and the like.

In embodiments, the target antigen or epitope used in antigen/receptor traps can include molecules from solid tumors and/or cancers. In embodiments, the target antigen or epitope used in antigen/receptor traps can include C-Met, CAIX, CD133, CD171, CD70, CEA, EGFR, EGFR vIII, Ep-CAM, EphA2, FAP, GD2, GPC3, HER2, HPV16-E6, IL13Ra2, LeY, MAGEA3, MAGEA4, MART1, Mesothelin, MUC1, MUC16, NY-ESO-1, PD-L1, PSCA, PSMA, ROR1, VEGFR2 and the like.

In some embodiments, an antigen/receptor trap molecule can regulate the cell-surface molecule (e.g., CAR) to which it binds. In some embodiments, the antigen/receptor trap can regulate the cell-surface molecule in cells in which the cell-surface molecule is overexpressed. In some embodiments, the antigen/receptor trap can up-regulate the cell-surface molecule in cells in which the cell-surface molecule is overexpressed.

In some embodiments, a dimerization means can be part of the CAR-Trap molecule. The dimerization means can be any region that can associate with another dimerization region, generally in a separate CAR-Trap molecule, using covalent or non-covalent bonds. The dimerized CAR-Trap molecules can be homodimers or heterodimers. There are many protein dimerization domains known in the art (e.g., see Dang, Dung Thanh. “Molecular Approaches to Protein Dimerization: Opportunities for Supramolecular Chemistry.” Frontiers in Chemistry 10 (2022): 829312). An example dimerization domain can include zipper motifs, like a leucine zipper.

In some embodiments, the dimerization means can be an Fc region from an antibody can be fused to the ectodomain. In some embodiments, the Fc region from IgG, IgM, IgA, IgE or IgD. In some embodiments, an Fc region can be (SEQ ID NO: 25):

DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK

In some embodiments, the Fc regions can dimerize, forming homodimers or heterodimer structures. In some embodiments, the Fc regions can have, or can be modified to have, cysteine amino acids that are capable of forming disulfide bonds (1 or more, such as 2 disulfide bonds). In some embodiments, dimers of the CAR-Traps that have Fc regions can form through disulfide bonds between cysteine residues in separate CAR-Trap molecules.

In certain embodiments, the Fc region can be a variant comprising an amino acid substitution which alters antigen-independent effector functions, like the circulating half-life of a molecule to which it is linked. Molecules linked to these Fc regions (e.g., an ectodomain) can exhibit either increased or decreased binding to FcRn compared to Fc regions lacking these substitutions, and can have an increased or decreased half-life in serum, respectively. Fc variants with improved affinity for FcRn are anticipated to have longer serum half-lives, and such molecules have useful applications in methods long half-life of the linked molecule is desired. In contrast, Fc variants with decreased FcRn binding affinity are expected to have shorter half-lives, and such molecules are also useful, for example, where a shortened circulation time can be advantageous. Fc variants with decreased FcRn binding affinity are also less likely to cross the placenta. In addition, other applications in which reduced FcRn binding affinity can be desired include those applications in which localization to the brain, kidney, and/or liver is desired. In one embodiment, the Fc variant-linked molecules can exhibit reduced transport across the epithelium of kidney glomeruli from the vasculature.

In another embodiment, the Fc variant-linked CAR-Trap molecules can exhibit reduced transport across the blood brain barrier (BBB) from the brain, into the vascular space. In one embodiment, an Fc region with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the bispecific modulators disclosed herein comprise an Fc domain having one or more of the following substitutions: V284E, H285E, N286D, K290E and S304D (EU numbering).

In some embodiments, an ectodomain disclosed herein can be linked to an Fc variant comprising an amino acid substitution which alters glycosylation. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the molecules can have an amino acid substitution near or within a glycosylation motif, for example, an N-linked glycosylation motif that contains the amino acid sequence NXT or NXS. In a particular embodiment, the Fc variant can have amino acid substitution at amino acid position 228 or 299 (EU numbering). Exemplary amino acid substitutions which confer reduced or altered glycosylation are described in PCT Publication No, WO05/018572, which is incorporated by reference herein in its entirety.

In some embodiments, the molecules disclosed herein can be modified to eliminate glycosylation and can be referred to as “agly” molecules. Exemplary agly molecules, can have an aglycosylated Fc region of an IgG4 antibody which is devoid of Fc-effector function thereby eliminating the potential for Fc mediated toxicity to the normal vital tissues and cells. In yet other embodiments, the molecules disclosed herein can have an altered glycan. For example, there can be a reduced number of fucose residues on an N-glycan at Asn297 of the Fc region, i.e., is afucosylated. In some embodiments, the there can be an altered number of sialic acid residues on the N-glycan at Asn297 of the Fc region.

In some embodiments, the CH2 or CH3 region of the Fc antibody domain can be truncated or modified to adjust the half-life of the molecule. In some embodiments, an Fc truncation includes CH3 or CH2 (e.g., Gehlsen, Kurt R., et al. “Pharmacokinetics of engineered human monomeric and dimeric CH2 domains.” MAbs. Vol. 4. No. 4. Taylor & Francis, 2012; Ying, Tianlei, et al. “Engineered soluble monomeric IgG1 CH3 domain: generation, mechanisms of function, and implications for design of biological therapeutics.” Journal of Biological Chemistry 288.35 (2013): 25154-25164).

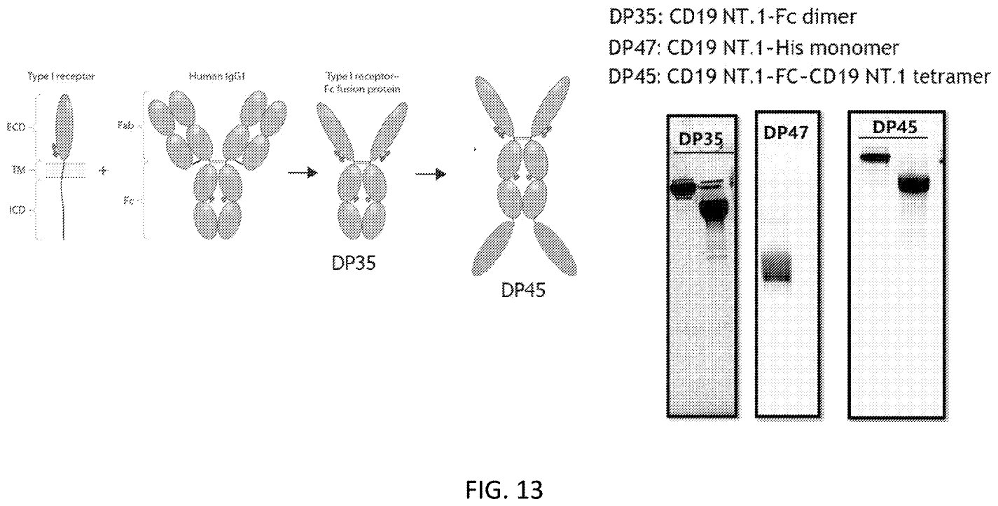

In some embodiments, the Fc regions can have, or can be modified to have, cysteine amino acids that are capable of forming disulfide bonds. In some embodiments, dimers or tetramers of the CAR-Trap molecules can form through disulfide bonds between cysteine residues in Fc regions of separate CAR-Trap molecules (e.g., FIG. 13, 28A or 29A). In some embodiments, other types of chemical bonds can form to obtain the multimeric molecules. In some embodiments, the bonds can form between regions of the CAR-Trap molecules that are not Fc regions. These dimers can be homodimers. In some embodiments, heterodimers can form. These multimers can have multiple ectodomains (e.g., can be multivalent for an ectodomain). In some embodiments, these CAR-Trap molecules can have 2, 3, 4, 5, 6, 7, 8 or more ectodomains.

In some embodiments, a linkage (e.g., linker) can be located between different sections of the CAR-Trap molecule. In some embodiments, this linkage can be located between the ectodomain or ectodomain means and a dimerization means. In some embodiments, the linkage can be a glycine-rich linker (“GS linker). In some embodiments, a “GS” linker can be a combination of glycine and serine amino acids. In some embodiments, the GS linker can be GSSGGSGGSGGS (SEQ ID NO: 28). Other sequences are possible. In some embodiments, the GS linker can be SGGGG (SEQ ID NO: 29), SGGGSGGG (SEQ ID NO: 30), GSSGGSGGSGGS (SEQ ID NO: 31), GSGS (SEQ ID NO: 32), GSGGS (SEQ ID NO: 33), GSSGSS (SEQ ID NO: 34), GSSSSSS (SEQ ID NO: 35) and the like. In some embodiments, a GS linker can have at least 4 amino acids that are glycine and/or serine. In some embodiments, other amino acids can be part of a GS linker, as long as glycine and serine are in the majority.

In some embodiments, the antigen/receptor trap molecules disclosed herein can include the following nucleotide and amino acid sequences, and molecules at least 60, 65, 70, 75, 80, 81, 82, 83, 84, 85, 86, 87, 88, 89, 90, 91, 92, 93, 94, 95, 96, 97, 98 or 99 percent identical to the nucleotide and amino acid sequences below. Specifically, the amino acid sequences of the antigen/receptor trap molecules can be labeled as follows:

pDP10 CD19-WT FC (SEQ ID NO: 1)
atgcgAatgcagctgctgctgctgattgcgctgagcctggcgctggtgaccaacagcactagtcccgaggaacctctagtg
gtgaaggtggaagagggagataacgctgtgctgcagtgcctcaaggggacctcagatggccccactcagcagctgacctggtctcggga
gtccccgcttaaacccttcttaaaactcagcctggggctgccaggcctgggaatccacatgaggcccctggccatctggcttttcatcttcaa
cgtctctcaacagatggggggcttctacctgtgccagccggggcccccctctgagaaggcctggcagcctggctggacagtcaatgtgga
gggcagcggggagctgttccggtggaatgtttcggacctaggtggcctgggctgtggcctgaagaacaggtcctcagagggccccagct
ccccttccgggaagctcatgagccccaagctgtatgtgtgggccaaagaccgccctgagatctgggagggagagcctccgtgtctcccac
cgagggacagcctgaaccagagcctcagccaggacctcaccatggcccctggctccacactctggctgtcctgtggggtaccccctgact
ctgtgtccaggggccccctctcctggacccatgtgcaccccaaggggcctaagtcattgctgagcctagagctgaaggacgatcgcccgg
ccagagatatgtgggtaatggagacgggtctgttgttgccccgggccacagctcaagacgctggaaagtattattgtcaccgtggcaacctg
accatgtcattccacctggagatcactgctcggccagtactatggcactggctgctgaggactggtggctggaagactagtTCTGGTG
GTGGTGGTGAGAATCTGTACTTTCAGAGCTCGGGCGGAGGATCgggtggaggcgagcccaaatc
ttgtgacaaaactcacacatgcCCCCCCTGCCCAGCGCCAGAATTGCTGGGCGGACCCAGCGTGT
TCCTGTTCCCCCCCAAACCTAAAGACACCCTGATGATCAGCCGAACCCCTGAGGTGA
CCTGCGTGGTGGTGGACGTGAGCCACGAGGACCCCGAGGTGAAGTTCAACTGGTAT
GTGGACGGCGTGGAGGTCCACAATGCCAAAACGAAGCCCAGGGAGGAGCAGTACA
ACAGCACCTACAGGGTAGTGAGCGTCTTGACCGTGCTGCACCAGGACTGGCTGAAC
GGCAAGGAATACAAATGCAAGGTCAGCAATAAGGCTCTGCCGGCTCCTATCGAGAA
GACAATCAGCAAGGCAAAGGGCCAGCCACGCGAACCGCAGGTGTATACTCTGCCCC
CCAGCCGGGACGAGCTGACCAAGAACCAGGTGTCCCTGACCTGTCTGGTGAAAGGC
TTCTACCCCAGCGACATCGCTGTGGAGTGGGAGAGTAACGGGCAGCCCGAGAACAA
CTACAAGACCACGCCTCCTGTGCTGGACAGCGACGGCAGCTTCTTCCTGTATAGCAA
GCTCACCGTGGACAAGAGCAGGTGGCAACAGGGCAACGTGTTCAGCTGCTCTGTGA
TGCACGAGGCCCTGCACAACCATTACACCCAGAAGAGTCTCAGTCTGAGCCCGGGA
AAGGGTGGAGGCGGATCCGGCCTGAACGACATCTTCGAGGCTCAGAAAATCGAATG
GCACGAAGGCtaa
(SEQ ID NO: 2)
MRMQLLLLIALSLALVTNSTSPEEPLVVKVEEGDNAVLQCLKGTSDGPTQQ
LTWSRESPLKPFLKLSLGLPGLGIHMRPLAIWLFIFNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRGNLTMS
FHLEITARPVLWHWLLRTGGWKTSSGGGGENLYFQSSGGGSGGGEPKSCDKTHTCPP
CPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGLNDIFEAQKIEWHEG*
pDP11 CD19-WT (SEQ ID NO: 3)
ATGCGAATGCAGCTGCTGCTGCTGATTGCGCTGAGCCTGGCGCTGGTGACC
AACAGCACTAGTcccgaggaacctctagtggtgaaggtggaagagggagataacgctgtgctgcagtgcctcaaggggacct
cagatggccccactcagcagctgacctggtctcgggagtccccgcttaaacccttcttaaaactcagcctggggctgccaggcctgggaat
ccacatgaggcccctggccatctggcttttcatcttcaacgtctctcaacagatggggggcttctacctgtgccagccggggcccccctctga
gaaggcctggcagcctggctggacagtcaatgtggagggcagcggggagctgttccggtggaatgtttcggacctaggtggcctgggct
gtggcctgaagaacaggtcctcagagggccccagctccccttccgggaagctcatgagccccaagctgtatgtgtgggccaaagaccgc
cctgagatctgggagggagagcctccgtgtctcccaccgagggacagcctgaaccagagcctcagccaggacctcaccatggcccctg
gctccacactctggctgtcctgtggggtaccccctgactctgtgtccaggggccccctctcctggacccatgtgcaccccaaggggcctaa
gtcattgctgagcctagagctgaaggacgatcgcccggccagagatatgtgggtaatggagacgggtctgttgttgccccgggccacagc
tcaagacgctggaaagtattattgtcaccgtggcaacctgaccatgtcattccacctggagatcactgctcggccagtactatggcactggct
gctgaggactggtggctggaagACTAGTTCTGGTGGTGGTGGTGGTGAGAATCTGTACTTTCAGA
GCTCGGGCGGAGGATCGGGTGGAGGCCACCACCATCATCACCACCATCACGGATCC
GGCCTGAACGACATCTTCGAGGCTCAGAAAATCGAATGGCACGAAGGCTAA
(SEQ ID NO: 4)
MRMQLLLLIALSLALVTNSTSPEEPLVVKVEEGDNAVLQCLKGTSDGPTQQ
LTWSRESPLKPFLKLSLGLPGLGIHMRPLAIWLFIFNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRGNLTMS
FHLEITARPVLWHWLLRTGGWKTSSGGGGGENLYFQSSGGGSGGGHHHHHHHHGSGL
NDIFEAQKIEWHEG*
pDP32-CD19-FC-CD19 WT (SEQ ID NO: 5)
atgcgAatgcagctgctgctgctgattgcgctgagcctggcgctggtgaccaacagcactagtcccgaggaacctctagtg
gtgaaggtggaagagggagataacgctgtgctgcagtgcctcaaggggacctcagatggccccactcagcagctgacctggtctcggga
gtccccgcttaaacccttcttaaaactcagcctggggctgccaggcctgggaatccacatgaggcccctggccatctggcttttcatcttcaa
cgtctctcaacagatggggggcttctacctgtgccagccggggcccccctctgagaaggcctggcagcctggctggacagtcaatgtgga
gggcagcggggagctgttccggtggaatgtttcggacctaggtggcctgggctgtggcctgaagaacaggtcctcagagggccccagct
ccccttccgggaagctcatgagccccaagctgtatgtgtgggccaaagaccgccctgagatctgggagggagagcctccgtgtctcccac
cgagggacagcctgaaccagagcctcagccaggacctcaccatggcccctggctccacactctggctgtcctgtggggtaccccctgact
ctgtgtccaggggccccctctcctggacccatgtgcaccccaaggggcctaagtcattgctgagcctagagctgaaggacgatcgcccgg
ccagagatatgtgggtaatggagacgggtctgttgttgccccgggccacagctcaagacgctggaaagtattattgtcaccgtggcaacctg
accatgtcattccacctggagatcactgctcggccagtactatggcactggctgctgaggactggtggctggaagactagtTCTGGTG
GTGGTGGTGAGAATCTGTACTTTCAGAGCTCGGGCGGAGGATCgggtggaggcgagcccaaatc
ttgtgacaaaactcacacatgcCCCCCCTGCCCAGCGCCAGAATTGCTGGGCGGACCCAGCGTGT
TCCTGTTCCCCCCCAAACCTAAAGACACCCTGATGATCAGCCGAACCCCTGAGGTGA
CCTGCGTGGTGGTGGACGTGAGCCACGAGGACCCCGAGGTGAAGTTCAACTGGTAT
GTGGACGGCGTGGAGGTCCACAATGCCAAAACGAAGCCCAGGGAGGAGCAGTACA
ACAGCACCTACAGGGTAGTGAGCGTCTTGACCGTGCTGCACCAGGACTGGCTGAAC
GGCAAGGAATACAAATGCAAGGTCAGCAATAAGGCTCTGCCGGCTCCTATCGAGAA
GACAATCAGCAAGGCAAAGGGCCAGCCACGCGAACCGCAGGTGTATACTCTGCCCC
CCAGCCGGGACGAGCTGACCAAGAACCAGGTGTCCCTGACCTGTCTGGTGAAAGGC
TTCTACCCCAGCGACATCGCTGTGGAGTGGGAGAGTAACGGGCAGCCCGAGAACAA
CTACAAGACCACGCCTCCTGTGCTGGACAGCGACGGCAGCTTCTTCCTGTATAGCAA
GCTCACCGTGGACAAGAGCAGGTGGCAACAGGGCAACGTGTTCAGCTGCTCTGTGA
TGCACGAGGCCCTGCACAACCATTACACCCAGAAGAGTCTCAGTCTGAGCCCGGGA
AAGGGTGGAGGCGGATCCggaggtagcggtggttctGGAcccgaggaacctctagtggtgaaggtggaagagggag
ataacgctgtgctgcagtgcctcaaggggacctcagatggccccactcagcagctgacctggtctcgggagtccccgcttaaacccttctta
aaactcagcctggggctgccaggcctgggaatccacatgaggcccctggccatctggcttttcatcttcaacgtctctcaacagatgggggg
cttctacctgtgccagccggggcccccctctgagaaggcctggcagcctggctggacagtcaatgtggagggcagcggggagctgttcc
ggtggaatgtttcggacctaggtggcctgggctgtggcctgaagaacaggtcctcagagggccccagctccccttccgggaagctcatga
gccccaagctgtatgtgtgggccaaagaccgccctgagatctgggagggagagcctccgtgtctcccaccgagggacagcctgaaccag
agcctcagccaggacctcaccatggcccctggctccacactctggctgtcctgtggggtaccccctgactctgtgtccaggggccccctctc
ctggacccatgtgcaccccaaggggcctaagtcattgctgagcctagagctgaaggacgatcgcccggccagagatatgtgggtaatgga
gacgggtctgttgttgccccgggccacagctcaagacgctggaaagtattattgtcaccgtggcaacctgaccatgtcattccacctggaga
tcactgctcggccagtactatggcactggctgctgaggactggtggctggaagGGCCTGAACGACATCTTCGAGGCT
CAGAAAATCGAATGGCACGAAGGCtaa
(SEQ ID NO: 6)
MRMQLLLLIALSLALVTNSTSPEEPLVVKVEEGDNAVLQCLKGTSDGPTQQ
LTWSRESPLKPFLKLSLGLPGLGIHMRPLAIWLFIFNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSQDLTMAPGSTLWLSCGVPPDSVSRGPLSWT
HVHPKGPKSLLSLELKDDRPARDMWVMETGLLLPRATAQDAGKYYCHRGNLTMS
FHLEITARPVLWHWLLRTGGWKTSSGGGGENLYFQSSGGGSGGGEPKSCDKTHTCPP
CPAPELLGGPSVFLFPPKPKDTLMISRTPEVTCVVVDVSHEDPEVKENWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGSGGSGPEEPLVVKVEEGDNAVLQCL
KGTSDGPTQQLTWSRESPLKPFLKLSLGLPGLGIHMRPLAIWLFIFNVSQQMGGFY
LCQPGPPSEKAWQPGWTVNVEGSGELFRWNVSDLGGLGCGLKNRSSEGPSSPSGK
LMSPKLYVWAKDRPEIWEGEPPCLPPRDSLNQSLSQDLTMAPGSTLWLSCGVPPDS
VSRGPLSWTHVHPKGPKSLLSLELKDDRPARDMWVMETGLLLPRATAQDAGKYY
CHRGNLTMSFHLEITARPVLWHWLLRTGGWKGLNDIFEAQKIEWHEG*
pDP33_CD19.1_Fc (SEQ ID NO: 7)
atgcgAatgcagctgctgctgctgattgcgctgagcctggcgctggtgaccaacagcactagtCCCGAGGAACC
TCTAGTGGTGAAGGTGGAAGAGGGAGATGAGGCTTGGCTCCCCTGCCTCAAGGGGA
CCTCCGATGGCCCCACTCAGCAGCTGACCTGGTCTCGGGAGTCCCCGCTTAAGCCCT
TCCTAAAAGTGAGTTTCGGGGTGCCAGGACTGGGCGTCCACGTGAGGCCCAACGCC
GTCTCTCTTGTCATCTCTAACGTCTCTCAACAGATGGGGGGCTTCTACCTGTGCCAGC
CGGGGCCCCCCTCTGAGAAGGCCTGGCAGCCTGGCTGGACAGTCAATGTGGAGGGC
AGCGGGGAGCTGTTCCGGTGGAATGTTTCGGACCTAGGTGGCCTGGGCTGTGGCCTG
AAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTC
TCCCACCGAGGGACAGCCTGAACCAGAGCCTCAGCAGGGACATGACTGTGGCCCCT
GGCTCCACACTCTGGCTGTCCTGTGGGGTACCCCCTGACTCTGTGTCCAGGGGCCCC
CTCTCCTGGACCCATGTGCACCCCAAGGGGCCTAAGTCATTGCTGAGCCTAGAGCTG
AAGGACGATCGCCCGGCCAGAGATATGTGGGTAACTGGTACTCGGCTGTTTCTGCCT
CGGGCCACAGCTCAAGACGCTGGAAAGTATTATTGCCACCGTGGCAACCTGACCAT
GTCATTCCACCTGGAGGTCAAAGCTCGGCCAGTCTCTGCTCACACTAAACTAAGAAC
TGGTGGCTGGAAGactagtTCTGGTGGTGGTGGTGAGAATCTGTACTTTCAGAGCTCGG
GCGGAGGATCgggtggaggcgagcccaaatcttgtgacaaaactcacacatgcCCCCCCTGCCCAGCGCCAG
AATTGCTGGGCGGACCCAGCGTGTTCCTGTTCCCCCCCAAACCTAAAGACACCCTGA
TGATCAGCCGAACCCCTGAGGTGACCTGCGTGGTGGTGGACGTGAGCCACGAGGAC
CCCGAGGTGAAGTTCAACTGGTATGTGGACGGCGTGGAGGTCCACAATGCCAAAAC
GAAGCCCAGGGAGGAGCAGTACAACAGCACCTACAGGGTAGTGAGCGTCTTGACCG
TGCTGCACCAGGACTGGCTGAACGGCAAGGAATACAAATGCAAGGTCAGCAATAAG
GCTCTGCCGGCTCCTATCGAGAAGACAATCAGCAAGGCAAAGGGCCAGCCACGCGA
ACCGCAGGTGTATACTCTGCCCCCCAGCCGGGACGAGCTGACCAAGAACCAGGTGT
CCCTGACCTGTCTGGTGAAAGGCTTCTACCCCAGCGACATCGCTGTGGAGTGGGAGA
GTAACGGGCAGCCCGAGAACAACTACAAGACCACGCCTCCTGTGCTGGACAGCGAC
GGCAGCTTCTTCCTGTATAGCAAGCTCACCGTGGACAAGAGCAGGTGGCAACAGGG
CAACGTGTTCAGCTGCTCTGTGATGCACGAGGCCCTGCACAACCATTACACCCAGAA
GAGTCTCAGTCTGAGCCCGGGAAAGGGTGGAGGCGGATCCGGCCTGAACGACATCT
TCGAGGCTCAGAAAATCGAATGGCACGAAGGCtaa
(SEQ ID NO: 8)
MRMQLLLLIALSLALVTNSTSPEEPLVVKVEEGDEAWLPCLKGTSDGPTQQ
LTWSRESPLKPFLKVSFGVPGLGVHVRPNAVSLVISNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSRDMTVAPGSTLWLSCGVPPDSVSRGPLSWT
HVHPKGPKSLLSLELKDDRPARDMWVTGTRLFLPRATAQDAGKYYCHRGNLTMS
FHLEVKARPVSAHTKLRTGGWKTSSGGGGENLYFQSSGGGSGGGEPKSCDKTHTCPPC
PAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGN
VFSCSVMHEALHNHYTQKSLSLSPGKGGGGSGLNDIFEAQKIEWHEG*
pDP34_CD19 C6.2-Fc (SEQ ID NO: 9)
atgcgAatgcagctgctgctgctgattgcgctgagcctggcgctggtgaccaacagcactagtcccgaggaacctctagtg
gtgaaggtggaagagggagataccgctgtgctgccttgcctcaaggggacctcagatggccccactcagcagctgacctggtctcggga
gtccccgcttaaacccttcttaaaatacagcctgggggtcccaggcctgggagtgcacgtgaggcccgatgccatctctgtggtgatccgg
aacgtctctcaacagatggggggcttctacctgtgccagccggggcccccctctgagaaggcctggcagcctggctggacagtcaatgtg
gagggcagcggggagctgttccggtggaatgtttcggacctaggtggcctgggctgtggcctgaagaacaggtcctcagagggccccag
ctccccttccgggaagctcatgagccccaagctgtatgtgtgggccaaagaccgccctgagatctgggagggagagcctccgtgtctccc
accgagggacagcctgaaccagagcctcagccaggacctcaccatggcccctggctccacactctggctgtcctgtggggtaccccctga
ctctgtgtccaggggccccctctcctggacccatgtgcaccccaaggggcctaagtcattgctgagcctagagctgaaggacgatcgccc
ggccagagagatgatcgtagatgagacgggtctgttgttgccccgggccacagctcaagacgctggaaagtggtattgttcacgtggcaac
gtaaccacctcatatcacctggagatcactgctcggccagtaaaggctcactcagacctgaggactggtggctggaagactagtTCTG
GTGGTGGTGGTGAGAATCTGTACTTTCAGAGCTCGGGCGGAGGATCgggtggaggcgagccc
aaatcttgtgacaaaactcacacatgcCCCCCCTGCCCAGCGCCAGAATTGCTGGGCGGACCCAGCG
TGTTCCTGTTCCCCCCCAAACCTAAAGACACCCTGATGATCAGCCGAACCCCTGAGG
TGACCTGCGTGGTGGTGGACGTGAGCCACGAGGACCCCGAGGTGAAGTTCAACTGG
TATGTGGACGGCGTGGAGGTCCACAATGCCAAAACGAAGCCCAGGGAGGAGCAGTA
CAACAGCACCTACAGGGTAGTGAGCGTCTTGACCGTGCTGCACCAGGACTGGCTGA
ACGGCAAGGAATACAAATGCAAGGTCAGCAATAAGGCTCTGCCGGCTCCTATCGAG
AAGACAATCAGCAAGGCAAAGGGCCAGCCACGCGAACCGCAGGTGTATACTCTGCC
CCCCAGCCGGGACGAGCTGACCAAGAACCAGGTGTCCCTGACCTGTCTGGTGAAAG
GCTTCTACCCCAGCGACATCGCTGTGGAGTGGGAGAGTAACGGGCAGCCCGAGAAC
AACTACAAGACCACGCCTCCTGTGCTGGACAGCGACGGCAGCTTCTTCCTGTATAGC
AAGCTCACCGTGGACAAGAGCAGGTGGCAACAGGGCAACGTGTTCAGCTGCTCTGT
GATGCACGAGGCCCTGCACAACCATTACACCCAGAAGAGTCTCAGTCTGAGCCCGG
GAAAGGGTGGAGGCGGATCCGGCCTGAACGACATCTTCGAGGCTCAGAAAATCGAA
TGGCACGAAGGCtaa
(SEQ ID NO: 10)
MRMQLLLLIALSLALVTNSTSPEEPLVVKVEEGDTAVLPCLKGTSDGPTQQ
LTWSRESPLKPFLKYSLGVPGLGVHVRPDAISVVIRNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSQDLTMAPGSTLWLSCGVPPDSVSRGPLSWT
HVHPKGPKSLLSLELKDDRPAREMIVDETGLLLPRATAQDAGKWYCSRGNVTTSY
HLEITARPVKAHSDLRTGGWKTSSGGGGENLYFQSSGGGSGGGEPKSCDKTHTCPPCP
APELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNV
FSCSVMHEALHNHYTQKSLSLSPGKGGGGSGLNDIFEAQKIEWHEG*
pDP35_CD19 NT.1-Fc (SEQ ID NO: 11)
atgcgAatgcagctgctgctgctgattgcgctgagcctggcgctggtgaccaacagcactagtcccgaggaacctctagtg
gtgaaggtggaagagggagataccgctgctctgtggtgcctcaaggggacctcagatggccccactcagcagctgacctggtctcggga
gtccccgcttaaacccttcttaaaatacagcctgggggtgccaggcctgggagtccacgtcaggcccgatgccatctctgtcgtcatcagga
acgtctctcaacagatggggggcttctacctgtgccagccggggcccccctctgagaaggcctggcagcctggctggacagtcaatgtgg
agggcagcggggagctgttccggtggaatgtttcggacctaggtggcctgggctgtggcctgaagaacaggtcctcagagggccccagc
tccccttccgggaagctcatgagccccaagctgtatgtgtgggccaaagaccgccctgagatctgggagggagagcctccgtgtctccca
ccgagggacagcctgaaccagagcctcagcagggacctcaccgtagcccctggctccacactctggctgtcctgtggggtaccccctga
ctctgtgtccaggggccccctctcctggacccatgtgcaccccaaggggcctaagtcattgctgagcctagagctgaaggacgatcgccc
ggccagagatatgtgggtaatgggtacgtcactgatgttgccccgggccacagctcaagacgctggaaagtggtattgtcaccgtggcaac
gtaaccacctcattccacctggaggtaatcgctcggccagtaaaggctcactcagacctgaggactggtggctggaagactagtTCTG
GTGGTGGTGGTGAGAATCTGTACTTTCAGAGCTCGGGCGGAGGATCgggtggaggcgagccc
aaatcttgtgacaaaactcacacatgcCCCCCCTGCCCAGCGCCAGAATTGCTGGGCGGACCCAGCG
TGTTCCTGTTCCCCCCCAAACCTAAAGACACCCTGATGATCAGCCGAACCCCTGAGG
TGACCTGCGTGGTGGTGGACGTGAGCCACGAGGACCCCGAGGTGAAGTTCAACTGG
TATGTGGACGGCGTGGAGGTCCACAATGCCAAAACGAAGCCCAGGGAGGAGCAGTA
CAACAGCACCTACAGGGTAGTGAGCGTCTTGACCGTGCTGCACCAGGACTGGCTGA
ACGGCAAGGAATACAAATGCAAGGTCAGCAATAAGGCTCTGCCGGCTCCTATCGAG
AAGACAATCAGCAAGGCAAAGGGCCAGCCACGCGAACCGCAGGTGTATACTCTGCC
CCCCAGCCGGGACGAGCTGACCAAGAACCAGGTGTCCCTGACCTGTCTGGTGAAAG
GCTTCTACCCCAGCGACATCGCTGTGGAGTGGGAGAGTAACGGGCAGCCCGAGAAC
AACTACAAGACCACGCCTCCTGTGCTGGACAGCGACGGCAGCTTCTTCCTGTATAGC
AAGCTCACCGTGGACAAGAGCAGGTGGCAACAGGGCAACGTGTTCAGCTGCTCTGT
GATGCACGAGGCCCTGCACAACCATTACACCCAGAAGAGTCTCAGTCTGAGCCCGG
GAAAGGGTGGAGGCGGATCCGGCCTGAACGACATCTTCGAGGCTCAGAAAATCGAA
TGGCACGAAGGCtaa
(SEQ ID NO: 12)
MRMQLLLLIALSLALVTNSTSPEEPLVVKVEEGDTAALWCLKGTSDGPTQQ
LTWSRESPLKPFLKYSLGVPGLGVHVRPDAISVVIRNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSRDLTVAPGSTLWLSCGVPPDSVSRGPLSWT
HVHPKGPKSLLSLELKDDRPARDMWVMGTSLMLPRATAQDAGKWYCHRGNVTT
SFHLEVIARPVKAHSDLRTGGWKTSSGGGGENLYFQSSGGGSGGGEPKSCDKTHTCPP
CPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGLNDIFEAQKIEWHEG*
pDP36_CD19 CT.2-Fc (SEQ ID NO: 13)
atgcgAatgcagctgctgctgctgattgcgctgagcctggcgctggtgaccaacagcactagtcccgaggaacctctagtg
gtgaaggtggaagagggagataccgctgctctgtggtgcctcaaggggacctcagatggccccactcagcagctgacctggtctcggga
gtccccgcttaaacccttcttaaaatatagcctgggggtcccaggcatgggagtccacgtcaggcccaacgccgtctctcttgtcatctctaa
cgtctctcaacagatggggggcttctacctgtgccagccggggcccccctctgagaaggcctggcagcctggctggacagtcaatgtgga
gggcagcggggagctgttccggtggaatgtttcggacctaggtggcctgggctgtggcctgaagaacaggtcctcagagggccccagct
ccccttccgggaagctcatgagccccaagctgtatgtgtgggccaaagaccgccctgagatctgggagggagagcctccgtgtctcccac
cgagggacagcctgaaccagagcctcagcagggacatgaccgtagcccctggctccacactctggctgtcctgtggggtaccccctgact
ctgtgtccaggggccccctctcctggacccatgtgcaccccaaggggcctaagtcattgctgagcctagagctgaaggacgatcgcccgg
ccagagatatgtgggtaatggagacgggtctggtattgccccgggccacagctcaagacgctggaaagtggtattgtcaccgtggcaacgt
aaccacttcatatcacctggagatcactgctcggccagtatcagctcacacccccctgaggactggtggctggaagactagtTCTGGT
GGTGGTGGTGAGAATCTGTACTTTCAGAGCTCGGGCGGAGGATCgggtggaggcgagcccaaa
tcttgtgacaaaactcacacatgcCCCCCCTGCCCAGCGCCAGAATTGCTGGGCGGACCCAGCGTG
TTCCTGTTCCCCCCCAAACCTAAAGACACCCTGATGATCAGCCGAACCCCTGAGGTG
ACCTGCGTGGTGGTGGACGTGAGCCACGAGGACCCCGAGGTGAAGTTCAACTGGTA
TGTGGACGGCGTGGAGGTCCACAATGCCAAAACGAAGCCCAGGGAGGAGCAGTAC
AACAGCACCTACAGGGTAGTGAGCGTCTTGACCGTGCTGCACCAGGACTGGCTGAA
CGGCAAGGAATACAAATGCAAGGTCAGCAATAAGGCTCTGCCGGCTCCTATCGAGA
AGACAATCAGCAAGGCAAAGGGCCAGCCACGCGAACCGCAGGTGTATACTCTGCCC
CCCAGCCGGGACGAGCTGACCAAGAACCAGGTGTCCCTGACCTGTCTGGTGAAAGG
CTTCTACCCCAGCGACATCGCTGTGGAGTGGGAGAGTAACGGGCAGCCCGAGAACA
ACTACAAGACCACGCCTCCTGTGCTGGACAGCGACGGCAGCTTCTTCCTGTATAGCA
AGCTCACCGTGGACAAGAGCAGGTGGCAACAGGGCAACGTGTTCAGCTGCTCTGTG
ATGCACGAGGCCCTGCACAACCATTACACCCAGAAGAGTCTCAGTCTGAGCCCGGG
AAAGGGTGGAGGCGGATCCGGCCTGAACGACATCTTCGAGGCTCAGAAAATCGAAT
GGCACGAAGGCtaa
(SEQ ID NO: 14)
MRMQLLLLIALSLALVTNSTSPEEPLVVKVEEGDTAALWCLKGTSDGPTQQ
LTWSRESPLKPFLKYSLGVPGMGVHVRPNAVSLVISNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSRDMTVAPGSTLWLSCGVPPDSVSRGPLSWT
HVHPKGPKSLLSLELKDDRPARDMWVMETGLVLPRATAQDAGKWYCHRGNVTT
SYHLEITARPVSAHTPLRTGGWKTSSGGGGENLYFQSSGGGSGGGEPKSCDKTHTCPP
CPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGLNDIFEAQKIEWHEG*
pDP45-CD19 NT.1-FC-CD19 NT.1 Tetramer (SEQ ID NO 15)
atgcgAatgcagctgctgctgctgattgcgctgagcctggcgctggtgaccaacagcactagtcccgaggaacctctagtg
gtgaaggtggaagagggagataccgctgctctgtggtgcctcaaggggacctcagatggccccactcagcagctgacctggtctcggga
gtccccgcttaaacccttcttaaaatacagcctgggggtgccaggcctgggagtccacgtcaggcccgatgccatctctgtcgtcatcagga
acgtctctcaacagatggggggcttctacctgtgccagccggggcccccctctgagaaggcctggcagcctggctggacagtcaatgtgg
agggcagcggggagctgttccggtggaatgtttcggacctaggtggcctgggctgtggcctgaagaacaggtcctcagagggccccagc
tccccttccgggaagctcatgagccccaagctgtatgtgtgggccaaagaccgccctgagatctgggagggagagcctccgtgtctccca
ccgagggacagcctgaaccagagcctcagcagggacctcaccgtagcccctggctccacactctggctgtcctgtggggtaccccctga
ctctgtgtccaggggccccctctcctggacccatgtgcaccccaaggggcctaagtcattgctgagcctagagctgaaggacgatcgccc
ggccagagatatgtgggtaatgggtacgtcactgatgttgccccgggccacagctcaagacgctggaaagtggtattgtcaccgtggcaac
gtaaccacctcattccacctggaggtaatcgctcggccagtaaaggctcactcagacctgaggactggtggctggaagactagtTCTG
GTGGTGGTGGTGAGAATCTGTACTTTCAGAGCTCGGGCGGAGGATCgggtggaggcgagccc
aaatcttgtgacaaaactcacacatgcCCCCCCTGCCCAGCGCCAGAATTGCTGGGCGGACCCAGCG
TGTTCCTGTTCCCCCCCAAACCTAAAGACACCCTGATGATCAGCCGAACCCCTGAGG
TGACCTGCGTGGTGGTGGACGTGAGCCACGAGGACCCCGAGGTGAAGTTCAACTGG
TATGTGGACGGCGTGGAGGTCCACAATGCCAAAACGAAGCCCAGGGAGGAGCAGTA
CAACAGCACCTACAGGGTAGTGAGCGTCTTGACCGTGCTGCACCAGGACTGGCTGA
ACGGCAAGGAATACAAATGCAAGGTCAGCAATAAGGCTCTGCCGGCTCCTATCGAG
AAGACAATCAGCAAGGCAAAGGGCCAGCCACGCGAACCGCAGGTGTATACTCTGCC
CCCCAGCCGGGACGAGCTGACCAAGAACCAGGTGTCCCTGACCTGTCTGGTGAAAG
GCTTCTACCCCAGCGACATCGCTGTGGAGTGGGAGAGTAACGGGCAGCCCGAGAAC
AACTACAAGACCACGCCTCCTGTGCTGGACAGCGACGGCAGCTTCTTCCTGTATAGC
AAGCTCACCGTGGACAAGAGCAGGTGGCAACAGGGCAACGTGTTCAGCTGCTCTGT
GATGCACGAGGCCCTGCACAACCATTACACCCAGAAGAGTCTCAGTCTGAGCCCGG
GAAAGGGTGGAGGCGGATCCggaggtagcggtggttctGGAcccgaggaacctctagtggtgaaggtggaagagg
gagataccgctgctctgtggtgcctcaaggggacctcagatggccccactcagcagctgacctggtctcgggagtccccgcttaaaccctt
cttaaaatacagcctgggggtgccaggcctgggagtccacgtcaggcccgatgccatctctgtcgtcatcaggaacgtctctcaacagatg
gggggcttctacctgtgccagccggggcccccctctgagaaggcctggcagcctggctggacagtcaatgtggagggcagcggggagc
tgttccggtggaatgtttcggacctaggtggcctgggctgtggcctgaagaacaggtcctcagagggccccagctccccttccgggaagct
catgagccccaagctgtatgtgtgggccaaagaccgccctgagatctgggagggagagcctccgtgtctcccaccgagggacagcctga
accagagcctcagcagggacctcaccgtagcccctggctccacactctggctgtcctgtggggtaccccctgactctgtgtccaggggccc
cctctcctggacccatgtgcaccccaaggggcctaagtcattgctgagcctagagctgaaggacgatcgcccggccagagatatgtgggt
aatgggtacgtcactgatgttgccccgggccacagctcaagacgctggaaagtggtattgtcaccgtggcaacgtaaccacctcattccacc
tggaggtaatcgctcggccagtaaaggctcactcagacctgaggactggtggctggaagGGCCTGAACGACATCTTCG
AGGCTCAGAAAATCGAATGGCACGAAGGCtaa
(SEQ ID NO: 16)
MRMQLLLLIALSLALVTNSTSPEEPLVVKVEEGDTAALWCLKGTSDGPTQQ
LTWSRESPLKPFLKYSLGVPGLGVHVRPDAISVVIRNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSRDLTVAPGSTLWLSCGVPPDSVSRGPLSWT
HVHPKGPKSLLSLELKDDRPARDMWVMGTSLMLPRATAQDAGKWYCHRGNVTT
SFHLEVIARPVKAHSDLRTGGWKTSSGGGGENLYFQSSGGGSGGGEPKSCDKTHTCPP
CPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGSGGSGPEEPLVVKVEEGDTAALWC
LKGTSDGPTQQLTWSRESPLKPFLKYSLGVPGLGVHVRPDAISVVIRNVSQQMGGF
YLCQPGPPSEKAWQPGWTVNVEGSGELFRWNVSDLGGLGCGLKNRSSEGPSSPSG
KLMSPKLYVWAKDRPEIWEGEPPCLPPRDSLNQSLSRDLTVAPGSTLWLSCGVPPD
SVSRGPLSWTHVHPKGPKSLLSLELKDDRPARDMWVMGTSLMLPRATAQDAGKW
YCHRGNVTTSFHLEVIARPVKAHSDLRTGGWKGLNDIFEAQKIEWHEG*
pDP47-CD19 NT.1_ Monomer (SEQ ID NO: 17)
ATGCGAATGCAGCTGCTGCTGCTGATTGCGCTGAGCCTGGCGCTGGTGACC
AACAGCACTAGTcccgaggaacctctagtggtgaaggtggaagagggagataccgctgctctgtggtgcctcaaggggacct
cagatggccccactcagcagctgacctggtctcgggagtccccgcttaaacccttcttaaaatacagcctgggggtgccaggcctgggagt
ccacgtcaggcccgatgccatctctgtcgtcatcaggaacgtctctcaacagatggggggcttctacctgtgccagccggggcccccctct
gagaaggcctggcagcctggctggacagtcaatgtggagggcagcggggagctgttccggtggaatgtttcggacctaggtggcctggg
ctgtggcctgaagaacaggtcctcagagggccccagctccccttccgggaagctcatgagccccaagctgtatgtgtgggccaaagaccg
ccctgagatctgggagggagagcctccgtgtctcccaccgagggacagcctgaaccagagcctcagcagggacctcaccgtagcccct
ggctccacactctggctgtcctgtggggtaccccctgactctgtgtccaggggccccctctcctggacccatgtgcaccccaaggggccta
agtcattgctgagcctagagctgaaggacgatcgcccggccagagatatgtgggtaatgggtacgtcactgatgttgccccgggccacag
ctcaagacgctggaaagtggtattgtcaccgtggcaacgtaaccacctcattccacctggaggtaatcgctcggccagtaaaggctcactca
gacctgaggactggtggctggaagACTAGTTCTGGTGGTGGTGGTGGTGAGAATCTGTACTTTCAG
AGCTCGGGCGGAGGATCGGGTGGAGGCCACCACCATCATCACCACCATCACGGATC
CGGCCTGAACGACATCTTCGAGGCTCAGAAAATCGAATGGCACGAAGGCTAA
(SEQ ID NO: 18)
MRMQLLLLIALSLALVTNSTSPEEPLVVKVEEGDTAALWCLKGTSDGPTQQ
LTWSRESPLKPFLKYSLGVPGLGVHVRPDAISVVIRNVSQQMGGFYLCQPGPPSEK
AWQPGWTVNVEGSGELFRWNVSDLGGLGCGLKNRSSEGPSSPSGKLMSPKLYVW
AKDRPEIWEGEPPCLPPRDSLNQSLSRDLTVAPGSTLWLSCGVPPDSVSRGPLSWT
HVHPKGPKSLLSLELKDDRPARDMWVMGTSLMLPRATAQDAGKWYCHRGNVTT
SFHLEVIARPVKAHSDLRTGGWKTSSGGGGGENLYFQSSGGGSGGGHHHHHHHHGSGL
NDIFEAQKIEWHEG*
pDP37-HER2 ETD_Fc (SEQ ID NO: 19)
atgcgAatgcagctgctgctgctgattgcgctgagcctggcgctggtgaccaacagcactagtACCCAAGTGTG
TACCGGAACAGATATGAAACTTCGGCTCCCAGCTTCCCCCGAGACTCACCTCGACAT
GTTGCGACATCTTTACCAGGGGtgccaggtggtgcagggaaacctggaactcacctacctgcccaccaatgccagcc
tgtccttcctgcaggatatccaggaggtgcagggctacgtgctcatcgctcacaaccaagtgaggcaggtcccactgcagaggctgcggat
tgtgcgaggcacccagctctttgaggacaactatgccctggccgtgctagacaatggagacccgctgaacaataccacccctgtcacagg
ggcctccccaggaggcctgcgggagctgcagcttcgaagcctcacagagatcttgaaaggaggggtcttgatccagcggaacccccagc
tctgctaccaggacacgattttgtggaaggacatcttccacaagaacaaccagctggctctcacactgatagacaccaaccgctctcgggcc
tgccacccctgttctccgatgtgtaagggctcccgctgctggggagagagttctgaggattgtcagagcctgacgcgcactgtctgtgccgg
tggctgtgcccgctgcaaggggccactgcccactgactgctgccatgagcagtgtgctgccggctgcacgggccccaagcactctgactg
cctggcctgcctccacttcaaccacagtggcatctgtgagctgcactgcccagccctggtcacctacaacacagacacgtttgagtccatgc
ccaatcccgagggccggtatacattcggcgccagctgtgtgactgcctgtccctacaactacctttctacggacgtgggatcctgcaccctc
gtctgccccctgcacaaccaagaggtgacagcagaggatggaacacagcggtgtgagaagtgcagcaagccctgtgcccgagtgtgcta
tggtctgggcatggagcacttgcgagaggtgagggcagttaccagtgccaatatccaggagtttgctggctgcaagaagatctttgggagc
ctggcatttctgccggagagctttgatggggacccagcctccaacactgccccgctccagccagagcagctccaagtgtttgagactctgg
aagagatcacaggttacctatacatctcagcatggccggacagcctgcctgacctcagcgtcttccagaacctgcaagtaatccggggacg
aattctgcacaatggcgcctactcgctgaccctgcaagggctgggcatcagctggctggggctgcgctcactgagggaactgggcagtgg
actggccctcatccaccataacacccacctctgcttcgtgcacacggtgccctgggaccagctctttcggaacccgcaccaagctctgctcc
acactgccaaccggccagaggacgagtgtgtgggcgagggcctggcctgccaccagctgtgcgcccgagggcactgctggggtccag
ggcccacccagtgtgtcaactgcagccagttccttcggggccaggagtgcgtggaggaatgccgagtactgcaggggctccccaggga
gtatgtgaatgccaggcactgtttgccgtgccaccctgagtgtcagccccagaatggctcagtgacctgttttggaccggaggctgaccagt
gtgtggcctgtgcccactataaggaccctcccttctgcgtggcccgctgccccagcggtgtgaaacctgacctctcctacatgcccatctgg
aagtttccagatgaggagggcgcatgccagccttgccccatcaactgcacccactcctgtgtggacctggatgacaagggctgccccgcc
gagcagagagccagccctctgacgactagtTCTGGTGGTGGTGGTGAGAATCTGTACTTTCAGAGCTC
GGGCGGAGGATCgggtggaggcgagcccaaatcttgtgacaaaactcacacatgcCCCCCCTGCCCAGCGCC
AGAATTGCTGGGCGGACCCAGCGTGTTCCTGTTCCCCCCCAAACCTAAAGACACCCT
GATGATCAGCCGAACCCCTGAGGTGACCTGCGTGGTGGTGGACGTGAGCCACGAGG
ACCCCGAGGTGAAGTTCAACTGGTATGTGGACGGCGTGGAGGTCCACAATGCCAAA
ACGAAGCCCAGGGAGGAGCAGTACAACAGCACCTACAGGGTAGTGAGCGTCTTGAC
CGTGCTGCACCAGGACTGGCTGAACGGCAAGGAATACAAATGCAAGGTCAGCAATA
AGGCTCTGCCGGCTCCTATCGAGAAGACAATCAGCAAGGCAAAGGGCCAGCCACGC
GAACCGCAGGTGTATACTCTGCCCCCCAGCCGGGACGAGCTGACCAAGAACCAGGT
GTCCCTGACCTGTCTGGTGAAAGGCTTCTACCCCAGCGACATCGCTGTGGAGTGGGA
GAGTAACGGGCAGCCCGAGAACAACTACAAGACCACGCCTCCTGTGCTGGACAGCG
ACGGCAGCTTCTTCCTGTATAGCAAGCTCACCGTGGACAAGAGCAGGTGGCAACAG
GGCAACGTGTTCAGCTGCTCTGTGATGCACGAGGCCCTGCACAACCATTACACCCAG
AAGAGTCTCAGTCTGAGCCCGGGAAAGGGTGGAGGCGGATCCGGCCTGAACGACAT
CTTCGAGGCTCAGAAAATCGAATGGCACGAAGGCtaa
(SEQ ID NO: 20)
MRMQLLLLIALSLALVTNSTSTQVCTGTDMKLRLPASPETHLDMLRHLYQ
GCQVVQGNLELTYLPTNASLSFLQDIQEVQGYVLIAHNQVRQVPLQRLRIVRGTQL
FEDNYALAVLDNGDPLNNTTPVTGASPGGLRELQLRSLTEILKGGVLIQRNPQLCY
QDTILWKDIFHKNNQLALTLIDTNRSRACHPCSPMCKGSRCWGESSEDCQSLTRTV
CAGGCARCKGPLPTDCCHEQCAAGCTGPKHSDCLACLHFNHSGICELHCPALVTY
NTDTFESMPNPEGRYTFGASCVTACPYNYLSTDVGSCTLVCPLHNQEVTAEDGTQR
CEKCSKPCARVCYGLGMEHLREVRAVTSANIQEFAGCKKIFGSLAFLPESFDGDPA
SNTAPLQPEQLQVFETLEEITGYLYISAWPDSLPDLSVFQNLQVIRGRILHNGAYSLT
LQGLGISWLGLRSLRELGSGLALIHHNTHLCFVHTVPWDQLFRNPHQALLHTANR
PEDECVGEGLACHQLCARGHCWGPGPTQCVNCSQFLRGQECVEECRVLQGLPRE
YVNARHCLPCHPECQPQNGSVTCFGPEADQCVACAHYKDPPFCVARCPSGVKPDL
SYMPIWKFPDEEGACQPCPINCTHSCVDLDDKGCPAEQRASPLTTSSGGGGENLYFQ
SSGGGSGGGEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGKGGGGSGLNDIFEA
QKIEWHEG*
pDP38-Herceptin BD-FC (SEQ ID NO: 21)
atgcgAatgcagctgctgctgctgattgcgctgagcctggcgctggtgaccaacagcactagtgtcaactgcagccagttc
cttcggggccaggagtgcgtggaggaatgccgagtactgcaggggctccccagggagtatgtgaatgccaggcactgtttgccgtgcca
ccctgagtgtcagccccagaatggctcagtgacctgttttggaccggaggctgaccagtgtgtggcctgtgcccactataaggaccctccct
tctgcgtggcccgctgccccagcggtgtgaaacctgacctctcctacatgcccatctggaagtttccagatgaggagggcgcatgccagcc
tactagtTCTGGTGGTGGTGGTGAGAATCTGTACTTTCAGAGCTCGGGCGGAGGATCgggt
ggaggcgagcccaaatcttgtgacaaaactcacacatgcCCCCCCTGCCCAGCGCCAGAATTGCTGGGCGG
ACCCAGCGTGTTCCTGTTCCCCCCCAAACCTAAAGACACCCTGATGATCAGCCGAAC
CCCTGAGGTGACCTGCGTGGTGGTGGACGTGAGCCACGAGGACCCCGAGGTGAAGT
TCAACTGGTATGTGGACGGCGTGGAGGTCCACAATGCCAAAACGAAGCCCAGGGAG
GAGCAGTACAACAGCACCTACAGGGTAGTGAGCGTCTTGACCGTGCTGCACCAGGA
CTGGCTGAACGGCAAGGAATACAAATGCAAGGTCAGCAATAAGGCTCTGCCGGCTC
CTATCGAGAAGACAATCAGCAAGGCAAAGGGCCAGCCACGCGAACCGCAGGTGTAT
ACTCTGCCCCCCAGCCGGGACGAGCTGACCAAGAACCAGGTGTCCCTGACCTGTCTG
GTGAAAGGCTTCTACCCCAGCGACATCGCTGTGGAGTGGGAGAGTAACGGGCAGCC
CGAGAACAACTACAAGACCACGCCTCCTGTGCTGGACAGCGACGGCAGCTTCTTCCT
GTATAGCAAGCTCACCGTGGACAAGAGCAGGTGGCAACAGGGCAACGTGTTCAGCT
GCTCTGTGATGCACGAGGCCCTGCACAACCATTACACCCAGAAGAGTCTCAGTCTGA
GCCCGGGAAAGGGTGGAGGCGGATCCGGCCTGAACGACATCTTCGAGGCTCAGAAA
ATCGAATGGCACGAAGGCtaa
(SEQ ID NO: 22)
MRMQLLLLIALSLALVTNSTSVNCSQFLRGQECVEECRVLQGLPREYVNAR
HCLPCHPECQPQNGSVTCFGPEADQCVACAHYKDPPFCVARCPSGVKPDLSYMPI
WKFPDEEGACQPTSSGGGGENLYFQSSGGGSGGGEPKSCDKTHTCPPCPAPELLGGPSVF
LFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALH
NHYTQKSLSLSPGKGGGGSGLNDIFEAQKIEWHEG*
pDP39-BCMA ECD-FC (SEQ ID NO: 23)
atgcgAatgcagctgctgctgctgattgcgctgagcctggcgctggtgaccaacagcactagtttgcagatggctgggcag
tgctcccaaaatgaatattttgacagtttgttgcatgcttgcataccttgtcaacttcgatgttcttctaatactcctcctctaacatgtcag
cgttattgtaatgcaagtgtgaccaattcagtgaaaggaacgaatgcgactagtTCTGGTGGTGGTGGTGAGAATCTGTACT
TTCAGAGCTCGGGCGGAGGATCgggtggaggcgagcccaaatcttgtgacaaaactcacacatgcCCCCCCT
GCCCAGCGCCAGAATTGCTGGGCGGACCCAGCGTGTTCCTGTTCCCCCCCAAACCTA
AAGACACCCTGATGATCAGCCGAACCCCTGAGGTGACCTGCGTGGTGGTGGACGTG
AGCCACGAGGACCCCGAGGTGAAGTTCAACTGGTATGTGGACGGCGTGGAGGTCCA
CAATGCCAAAACGAAGCCCAGGGAGGAGCAGTACAACAGCACCTACAGGGTAGTG
AGCGTCTTGACCGTGCTGCACCAGGACTGGCTGAACGGCAAGGAATACAAATGCAA
GGTCAGCAATAAGGCTCTGCCGGCTCCTATCGAGAAGACAATCAGCAAGGCAAAGG
GCCAGCCACGCGAACCGCAGGTGTATACTCTGCCCCCCAGCCGGGACGAGCTGACC
AAGAACCAGGTGTCCCTGACCTGTCTGGTGAAAGGCTTCTACCCCAGCGACATCGCT
GTGGAGTGGGAGAGTAACGGGCAGCCCGAGAACAACTACAAGACCACGCCTCCTGT
GCTGGACAGCGACGGCAGCTTCTTCCTGTATAGCAAGCTCACCGTGGACAAGAGCA
GGTGGCAACAGGGCAACGTGTTCAGCTGCTCTGTGATGCACGAGGCCCTGCACAAC
CATTACACCCAGAAGAGTCTCAGTCTGAGCCCGGGAAAGGGTGGAGGCGGATCCGG
CCTGAACGACATCTTCGAGGCTCAGAAAATCGAATGGCACGAAGGCtaa
(SEQ ID NO: 24)
MRMQLLLLIALSLALVTNSTSLQMAGQCSQNEYFDSLLHACIPCQLRCSSNT
PPLTCQRYCNASVTNSVKGTNATSSGGGGENLYFQSSGGGSGGGEPKSCDKTHTCPPCP
APELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNV
FSCSVMHEALHNHYTQKSLSLSPGKGGGGSGLNDIFEAQKIEWHEG
Times New Roman font underlined is signal peptide,
Times New Roman font bolded is ectodomain;
Times New Roman font italicized is linker, in some embodiments encoded by
restriction enzyme site creation;
Times New Roman font underlined and bolded is GS linker;
Times New Roman font underlined, italicized, and bolded is cleavable linker;
Courier New font underlined is Fc region;
Courier New font bolded is TEV site;
Courier New font underlined and bolded is His-Tag; and
Courier New font underlined, italicized, and bolded is Avi-Tag.

### Antibodies

“Recombinant” as it pertains to polypeptides (such as antibodies) or polynucleotides refers to a form of the polypeptide or polynucleotide that does not exist naturally, a non-limiting example of which can be created by combining polynucleotides or polypeptides that would not normally occur together. “Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It may be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of the antibodies disclosed herein can exhibit increased cross-reactivity in comparison to an unmodified antibody.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

Some embodiments also feature antibodies that have a specified percentage identity or similarity to the amino acid or nucleotide sequences of the antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e., BLAST or BLAST 2.0), manual alignment or visual inspection can be utilized to determine percent sequence identity or similarity for the nucleic acids and proteins of the present invention.

Aspects of the invention provide isolated antibodies. The term “isolated” as used herein with respect to cells, nucleic acids, such as DNA or RNA, refers to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides.

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. “Specifically binds” or “immunoreacts with” can refer to the antibody reacting with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, F_{ab}′, F_{ab}, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (V_{H}) and light chains (V_{L}) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85(16):5879-5883). In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight approximately 53,000-70,000. The four chains are joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgAQ1 and IgA2) or subclass of an immunoglobulin molecule.

Light chains are classified as either kappa or lambda (κ, λ). Each heavy chain class can be bound with either a kappa or lambda light chain. For example, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less intermolecular variability. The framework regions largely adopt a β-sheet conformation and the CDRs form loops which connect, and in some cases form part of, the β-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR | Numbering | Numbering |
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and can have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e., CDR-H1, CDR-H2, CDR-H3, CDR-L1, CDR-L2 and CDR-L3).

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361: 186-87 (1993)). The ratio of K_{off}/Kₒₙ allows the cancellation of all parameters not related to affinity, and is equal to the equilibrium binding constant, K_{D}. (See, generally, Davies et al. (1990) Annual Rev Biochem 59:439-473). An antibody of the invention can specifically bind to an epitope when the equilibrium binding constant (K_{D}) is ≤1 μM, ≤10 μM, ≤10 nM, ≤10 pM, or ≤100 pM to about 1 pM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a Ko about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1F-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the antibody can be monovalent or bivalent, and can comprise a single or double chain. Functionally, the binding affinity of the antibody is within the range of 10⁻⁵ M to 10⁻¹² M. For example, the binding affinity of the antibody is from 10⁻⁶ M to 10⁻¹² M, from 10⁻⁷ M to 10⁻¹² M, from 10⁻⁸ M to 10⁻¹² M, from 10⁻⁹ M to 10⁻¹² M, from 10⁻⁵ M to 10⁻¹¹ M, from 10⁻⁶ M to 10⁻¹¹ M, from 10⁻⁷M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10⁻ M to 10⁻¹⁰ M, from 10⁻⁷M to 10⁻¹⁰ M, from 10⁻⁸ M to 10⁻¹⁰ M, from 10⁻⁹ M to 10⁻¹⁰ M, from 10⁻⁵ M to 10⁻⁹M, from 10⁻⁶ M to 10⁻⁹M, from 10⁻⁷ M to 10⁻⁹M, from 10⁻⁸ M to 10⁻⁹M, from 10⁻⁵ M to 10⁻⁸ M, from 10⁻⁶ M to 10⁻⁸ M, from 10⁻⁷M to 10⁻⁸ M, from 10⁻⁵ M to 10⁻⁷M, from 10⁻⁶ M to 10⁻⁷M, or from 10⁻⁵ M to 10⁻⁶ M.

Those skilled in the art will recognize that one can determine, without undue experimentation, if a human monoclonal antibody has the same specificity as a human monoclonal antibody of the invention by ascertaining whether the former prevents the latter from specifically binding. For example, if the human monoclonal antibody being tested competes with the human monoclonal antibody of the invention, as shown by a decrease in binding by the human monoclonal antibody of the invention, then the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with an epitope, with which it is normally reactive, and then add the human monoclonal antibody being tested to determine if the human monoclonal antibody being tested is inhibited in its ability to bind the epitope. If the human monoclonal antibody being tested is inhibited then, it has the same, or functionally equivalent, epitopic specificity as the monoclonal antibody of the invention. Screening of human monoclonal antibodies of the invention can be also carried out by utilizing epitopes and determining whether the test monoclonal antibody is able to neutralize polypeptides containing the epitope.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in all the molecules of the population. MAbs contain an antigen binding site that can immunoreact with a specific epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is immunized with an immunizing agent to elicit lymphocytes that produce or are capable of producing antibodies that will specifically bind to the immunizing agent. Alternatively, the lymphocytes can be immunized in vitro.

### Nucleic Acids, Vectors and Cells Expressing Receptor Traps

Also disclosed are nucleic acids encoding all or part of the receptor traps described herein. Also disclosed are various vectors (e.g., plasmids, viral, and the like) that include the nucleic acids. Also disclosed are various cells (e.g., prokaryotic, eukaryotic) that contain nucleic acids or vectors and can express the fusion proteins.

### Methods

Disclosed herein are methods for administrating the antigen/receptor traps described herein to a subject. In some embodiments, the antigen/receptor traps can target CAR receptors on CAR-T cells. In some embodiments, the methods are used to treat toxicities in subjects who have received CAR-T cell infusion for treatment of cancer (e.g., toxicities due to cytokine release) or to treat CAR-T cell exhaustion in like subjects. In some embodiments, the methods are used to improve efficacy of CAR-T cells that have been administered to a subject to treat cancer.

In some embodiments, the subject who is being treated for cancer can have received a CAR-T cell therapy for a cancer selected from the group consisting of B-cell acute lymphoblastic leukemia (ALL), B-cell non-Hodgkin lymphoma (NHL), follicular lymphoma, mantle cell lymphoma (MCL), multiple myeloma, and the like.

In some embodiments, the reagents and methods disclosed herein can be used with cells that are not cancer cells.

### Therapeutic Preparations

Aspects of this disclosure are drawn towards therapeutic preparations. As used herein, the term “therapeutic preparation” can refer to any compound or composition that can be used or administered for therapeutic effects (e.g., receptor traps). As used herein, the term “therapeutic effects” can refer to effects sufficient to result in amelioration of symptoms, e.g., treatment, healing, prevention or amelioration of the relevant medical condition, or an increase in rate of treatment, healing, prevention or amelioration of such conditions.

Embodiments as described herein can be administered to a subject in the form of a pharmaceutical composition or therapeutic preparation prepared for the intended route of administration. Such compositions and preparations can comprise, for example, the active ingredient(s) and a pharmaceutically acceptable carrier. Such compositions and preparations can be in a form adapted to oral, subcutaneous, parenteral (such as, intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions and preparations can appear in conventional forms, for example, solutions and suspensions for injection, capsules and tablets, in the form of enteric formulations, e.g., as disclosed in U.S. Pat. No. 5,350,741, and for oral administration.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition can be sterile and can be fluid to the extent that easy syringeability exists. In embodiments, it can be stable under the conditions of manufacture and storage and can be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by using a coating such as lecithin, by the maintenance of the required particle size in the case of dispersion and by using surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel® (sodium starch glycolate), or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

In embodiments, administering can comprise the placement of a pharmaceutical composition into a subject by a method or route which results in at least partial localization of the composition at a desired site such that desired effect is produced.

For example, the pharmaceutical composition can be administered by bolus injection or by infusion. A bolus injection can refer to a route of administration in which a syringe is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. Administration can continue for up to one year. In embodiments, administration can continue for the life of the subject. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration in a continuous manner, such as to maintain the therapeutic effect (activity) over a prolonged period of time.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of a reagent or therapeutic composition of the invention can be the amount needed to achieve a therapeutic objective. As noted herein, this can be a binding interaction between the reagent or therapeutic composition and its target that, in certain cases, interferes with the functioning of the target. The amount required to be administered will furthermore depend on the binding affinity of the reagent or therapeutic composition for its specific target and will also depend on the rate at which an administered reagent or therapeutic composition is depleted from the free volume other subject to which it is administered. The dosage administered to a subject (e.g., a patient) of the binding polypeptides described herein is about 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration is often possible. Further, the dosage and frequency of administration of reagent or therapeutic composition of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where fragments (e.g., antibody fragments) are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90:7889-7893 (1993)). The formulation can also contain more than one active compound as necessary for the specific indication being treated, for example, those with complementary activities that do not adversely affect each other. Alternatively, or in addition, the composition can comprise an agent that enhances its function, such as, for example, a cytotoxic agent, cytokine (e.g., IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles, and nanocapsules) or in macroemulsions. Sustained-released preparations can be prepared.

The pharmaceutical or therapeutic carrier or diluent employed can be a conventional solid or liquid carrier. Nonlimiting examples of solid carriers are lactose, terra alba, sucrose, cyclodextrin, talc, gelatin, agar, pectin, acacia, magnesium stearate, stearic acid or lower alkyl ethers of cellulose. Nonlimiting examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tableted, placed in a hard gelatin capsule in powder or pellet form or it can be in the form of a troche or lozenge. The amount of solid carrier will vary widely but can be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The composition and/or preparation can also be in a form suited for local or systemic injection or infusion and can, as such, be formulated with sterile water or an isotonic saline or glucose solution. The compositions can be in a form adapted for peripheral administration only, except for centrally administrable forms. The compositions and/or preparations can be in a form adapted for central administration.

The compositions and/or preparations can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The compositions and/or preparations can contain pharmaceutically and/or therapeutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

### EMBODIMENTS

Disclosed below in numbered paragraphs are example embodiments disclosed herein.

1. An antigen trap or receptor trap as disclosed herein.

2. The antigen or receptor trap of embodiment 1, comprising an antigen or epitope that is bound by a chimeric antigen receptor (CAR).

3. The antigen or receptor trap of embodiment 2, wherein the CAR is on a T cell (CAR-T cell).

4. The antigen or receptor trap of embodiment 3, wherein binding the antigen or trap by the CAR:
a. substantially blocks binding of other antigens or epitopes to which the CAR can bind; and/or
b. does not substantially activate or repress the CAR-T cell.

5. An antigen or epitope to which a CAR on a CAR-T cell can bind, wherein binding of the antigen by the CAR:
a. inhibits binding of the CAR to its cognate antigen displayed on a surface of a target cell; and/or
b. does not substantially activate or repress the CAR-T cell.

6. The antigen or epitope of embodiment 5, wherein the antigen or epitope is not associated with a cell.

7. The antigen or epitope of embodiment 5, additionally comprising an Fc region of an antibody fused to the antigen or epitope.

8. The antigen or epitope of embodiment 5, wherein the antigen or epitope is from a tumor-specific antigen (TSA) or tumor-associated antigen (TAA).

9. The antigen or epitope of embodiment 5, wherein the antigen or epitope is from a blood (hematological) cancer cell or a cell from a solid tumor.

10. The antigen or epitope of embodiment 5, wherein the antigen or epitope is from cells from a B-cell acute lymphoblastic leukemia (ALL), B-cell non-Hodgkin lymphoma (NHL), follicular lymphoma, mantle cell lymphoma (MCL) or multiple myeloma.

11. The antigen or epitope of embodiment 5, wherein the antigen or epitope is from cells from a brain tumor, breast tumor or kidney tumor.

12. The antigen or epitope of epitope 5, wherein the antigen or epitope is from cells from a hepatocellular carcinoma, GPC3 positive hepatocellular carcinoma, hepatic carcinoma, lung cancer, advanced lung cancer, advanced solid tumor, colon cancer, colorectal cancer, EGFR-positive colorectal cancer, esophageal carcinoma, pancreatic cancer, prostate cancer, gastric cancer, sarcoma, osteoid sarcoma, Ewing sarcoma, breast cancer, ovarian cancer, glioma, cervical cancer, squamous cell lung cancer, liver metastasis, liver neoplasm, stomach neoplasm, or advanced EGFR-positive solid tumor.

13. The antigen or epitope of embodiment 5, wherein the CAR comprises a CD19-specific, a human epidermal growth factor receptor 2 (HER2)-specific, or a B-cell maturation antigen (BMCA)-specific CAR.

14. The antigen or epitope of embodiment 5, wherein the antigen or epitope binds to a CAR specific for CD123, CD138, CD20, CD22, CD38, CD5, Ig κ chain, LeY, NKG2D ligand, ROR1, or WT1.

15. The antigen or epitope of embodiment 5, wherein the antigen or epitope binds to a CAR specific for C-Met, CAIX, CD133, CD171, CD70, CEA, EGFR, EGFR VIII, Ep-CAM, EphA2, FAP, GD2, GPC3, HER2, HPV16-E6, IL13Ra2, LeY, MAGEA3, MAGEA4, MART1, Mesothelin, MUC1, MUC16, NY-ESO-1, PD-L1, PSCA, PSMA, ROR1, or VEGFR2.

16. The antigen or epitope of embodiment 5, comprising an amino acid sequence SEQ ID NO: 2, 4, 6, 8, 10, 12, 14, 16, 18, 20, 22, 24, or an amino acid sequence at least 85, 86, 87, 88, 89, 90, 91, 92, 93, 94, 95, 96, 97, 98 or 99 percent identical thereto.

17. The antigen or epitope of embodiment 5, wherein a nucleotide sequence encoding the antigen or epitope comprises SEQ ID NO: 1, 3, 5, 7, 9, 11, 13, 15, 17, 19, 21, 23, or a nucleotide sequence at least 85, 86, 87, 88, 89, 90, 91, 92, 93, 94, 95, 96, 97, 98 or 99 percent identical thereto.

18. A nucleic acid encoding the antigen or epitope of embodiment 5.

19. A vector comprising the nucleic acid of embodiment 18.

20. A cell comprising the vector of embodiment 19.

21. A method for treating an ailment associated with CAR-T therapy, or for increasing efficacy of CAR-T therapy, comprising administering the antigen or epitope of any one of embodiments 5-17.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Receptor Traps and Expression

Soluble ectodomains of CAR antigens can prevent the interaction of CAR-T cells with the antigens on tumor cells. CD19 is expressed on normal and malignant B cells. In one embodiment, the ectodomain of CD19 was tested for use as a receptor trap.

We first used a full-length extracellular domain of CD19. In some embodiments, wildtype CD19 ectodomain can be difficult to express.

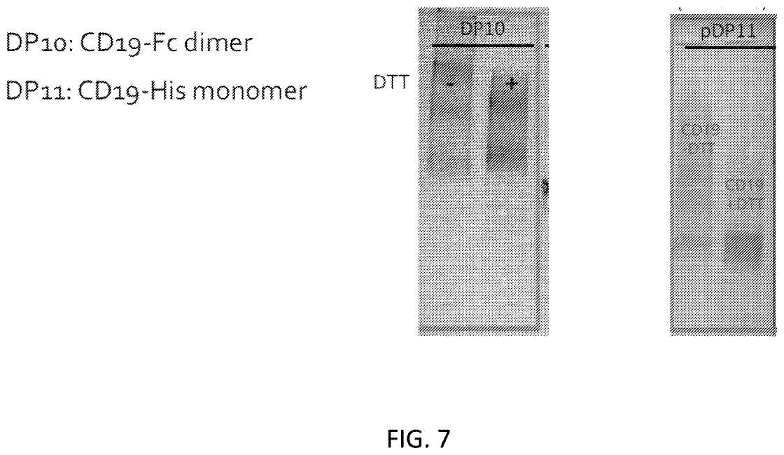

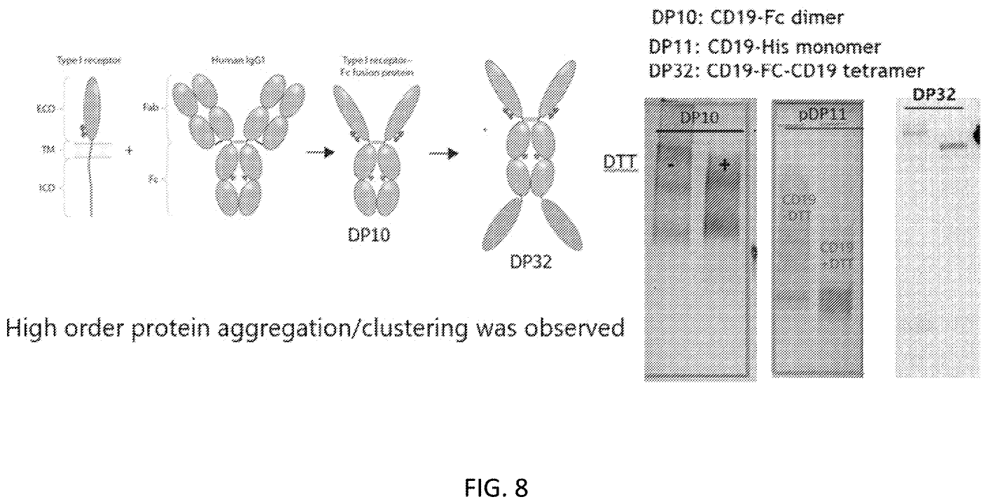

The ectodomain of CD19, described above, was expressed both as a monomer (CD19ecto) and as a Fc-fusion dimer (CD19ecto-Fc). In expression of these polypeptides, we observed higher order protein aggregation and/or clustering (FIG. 7). We also observed higher order aggregation/clustering when the Fc-fusion dimer was arranged as a tetramer (FIG. 8).

### Example 2—Function of Receptor Traps

To test the ability of the various receptor traps to affect CAR-T cell activity, we used a Jurkat cell expressing a chimeric antigen receptor (CAR) specific for the extracellular domain of CD19. The Jurkat cell line, when activated by binding to the extracellular domain of CD19 on a tumor cell, expressed GFP which was detectable using flow cytometry. We used a second cell line, K562, that expresses the extracellular domain of CD19 on its surface. In the assay, when the Jurkat CAR binds CD19 on the surface of the K562 cells, activation of the Jurkat cells is measured by expression of GFP in the Jurkat cells.

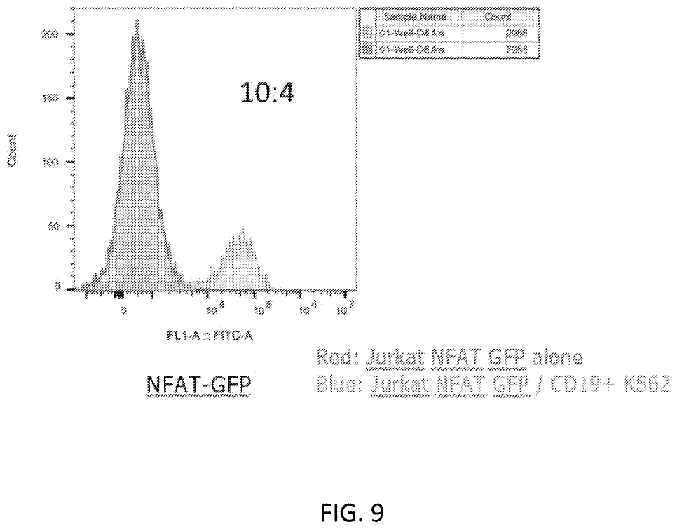

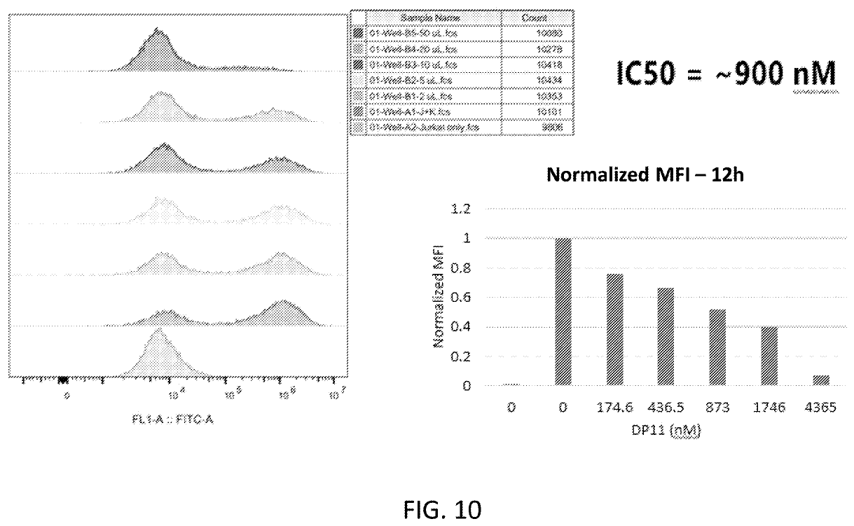

In the assay, inactivated Jurkat cells alone (without K562) produced a peak of background fluorescence (red peak in FIG. 9). When K562 cells were added, and the Jurkat CAR bound to the extracellular CD19 domain on the surface of the K562 cells, Jurkat cells were activated and could be detected as a fluorescent cell population (blue peak in FIG. 9) above the background fluorescence of the inactivated Jurkat cells (red peak).

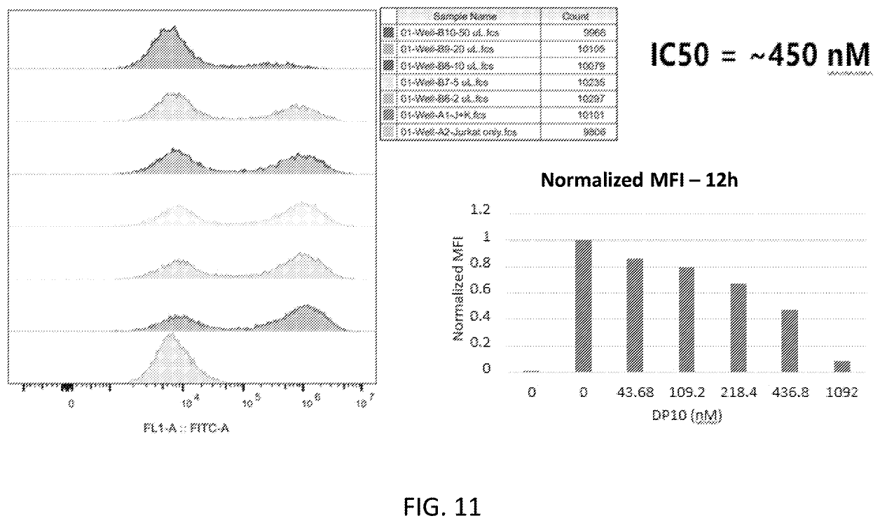

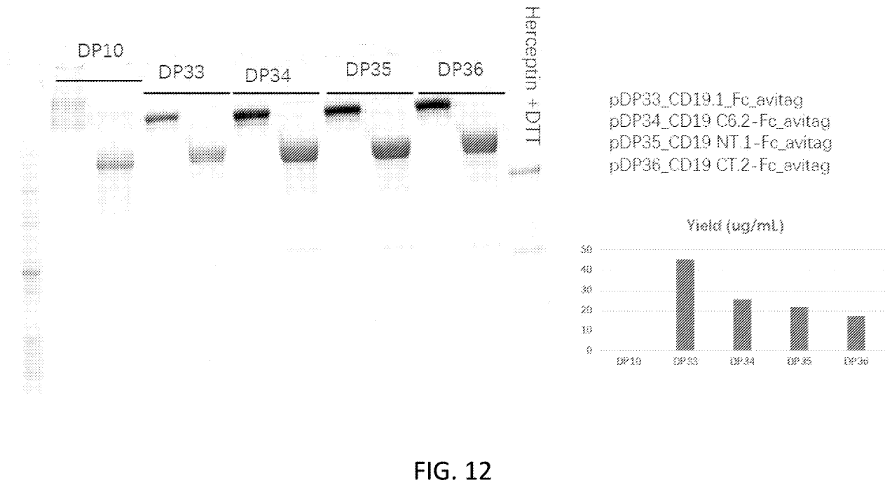

To test the receptor traps, the receptor traps were added to the combination of the Jurkat and K562 cells. In the absence of CD19ecto proteins, co-incubation of CD19+K562 leukemia cells and anti-CD19-CAR-Jurkat NFAT-GFP cells induced Jurkat activation (indicated by high GFP expression) (FIG. 9). Dose-dependent decrease of GFP fluorescence was observed with addition of the CD19ecto (FIG. 10) or CD19ecto-Fc proteins (FIG. 11), indicating these proteins can block CAR-T/tumor interaction. Additionally, removal of the proteins reversed the inhibition effect. Furthermore, both CD19ecto and CD19ecto-Fc showed neglectable activation of the CAR-Jurkat cells without the tumor cells.

In other studies, engineered CD19 variants were used to improve expression yield and reduce aggregation. Some variants of this type are described in Klesmith, Justin R., et al. “Retargeting CD19 chimeric antigen receptor T cells via engineered CD19-fusion proteins.” Molecular pharmaceutics 16.8 (2019): 3544-3558. One example of expression of variants of this type is shown in FIG. 12. The data showed that these variants improved CD19 expression and reduced aggregation (FIG. 13).

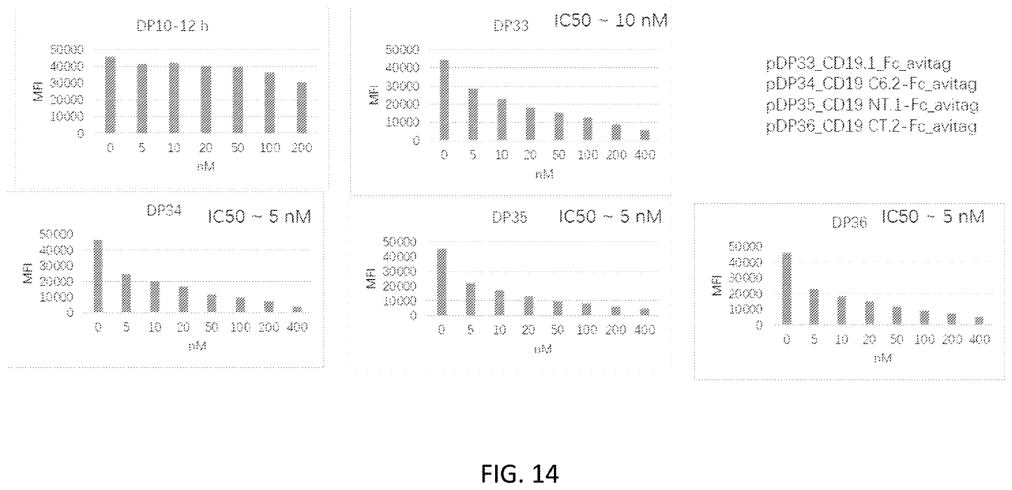

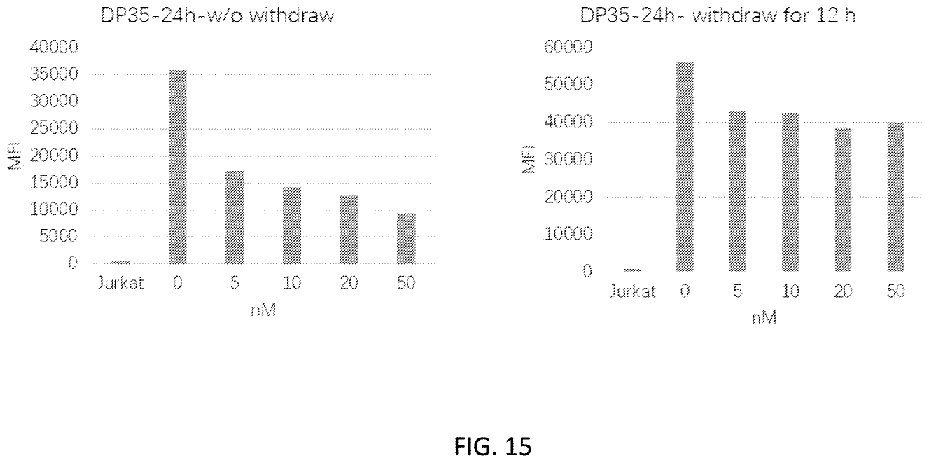

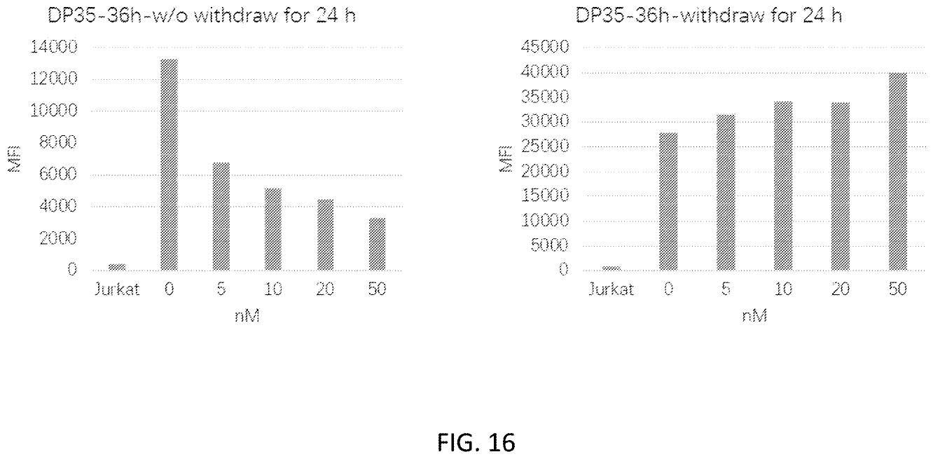

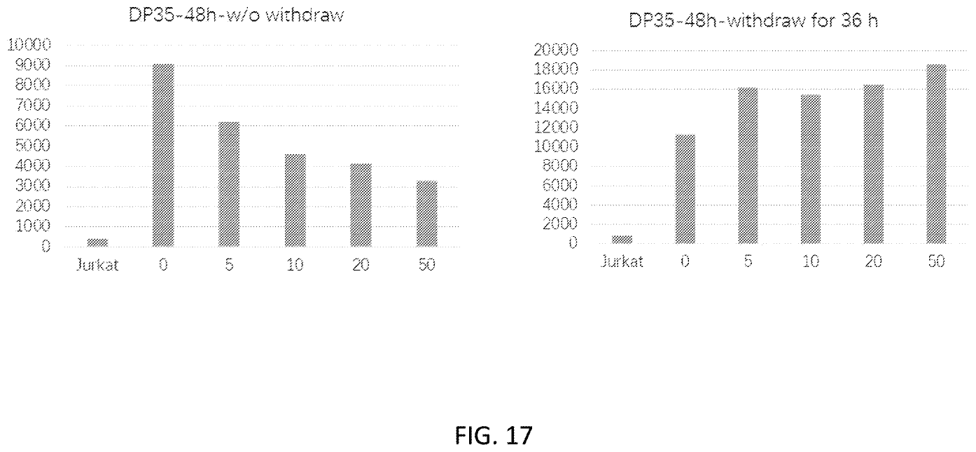

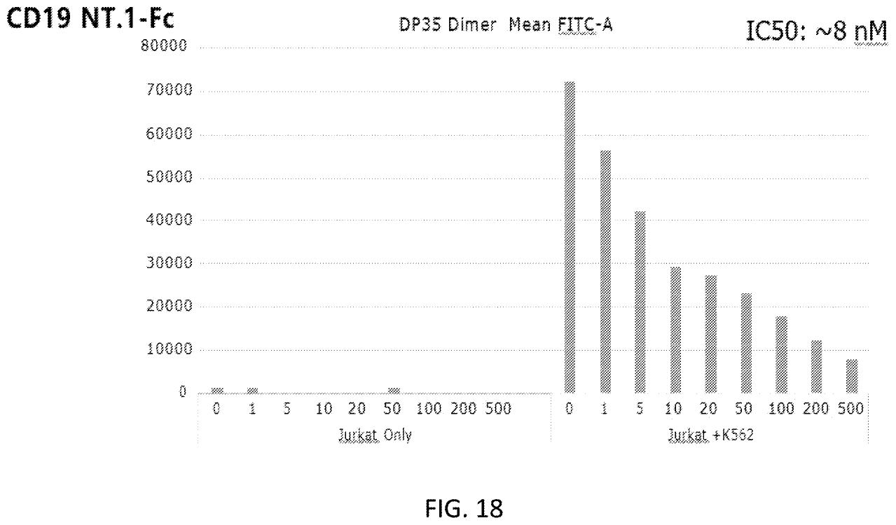

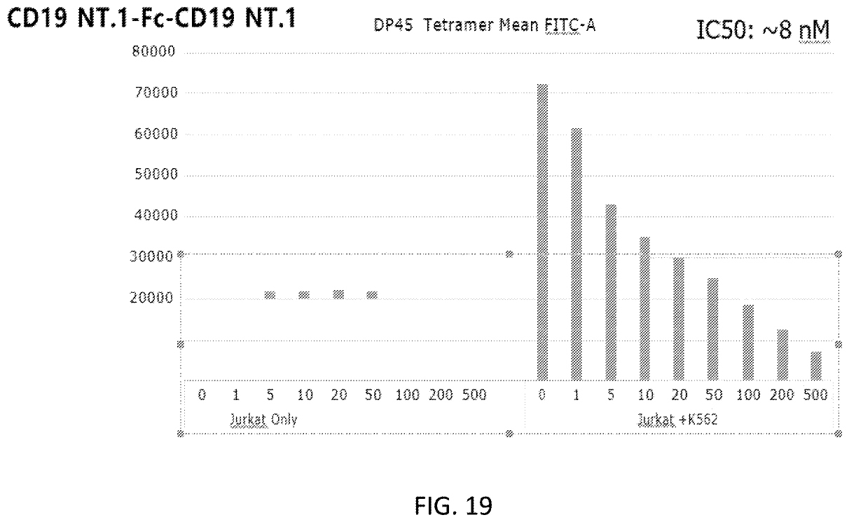

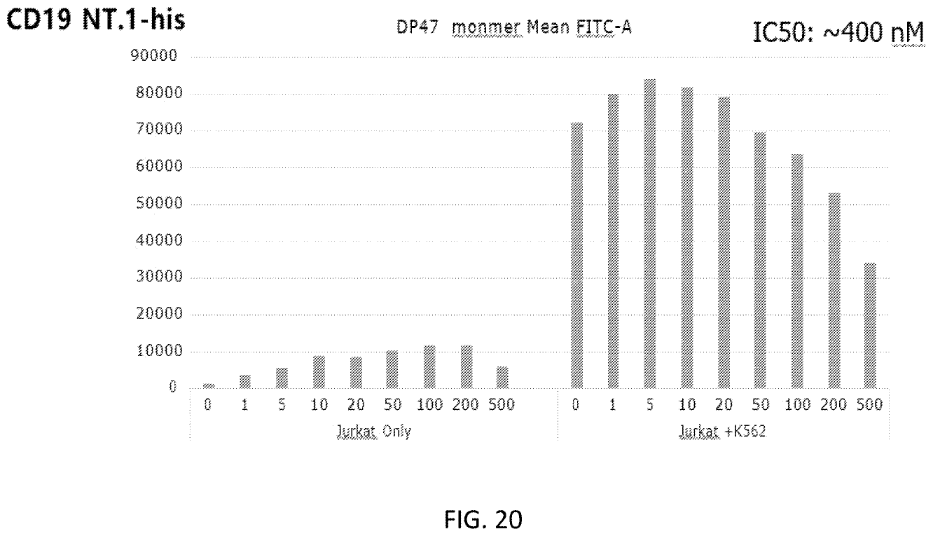

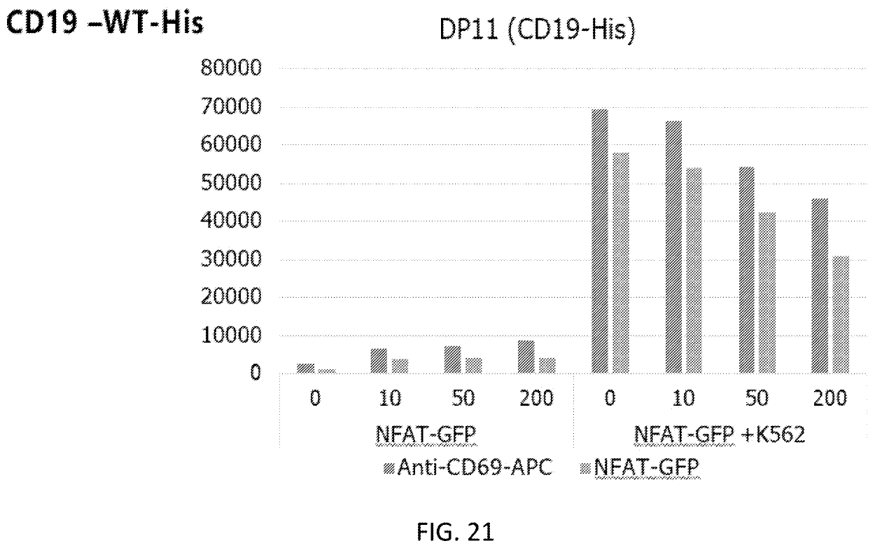

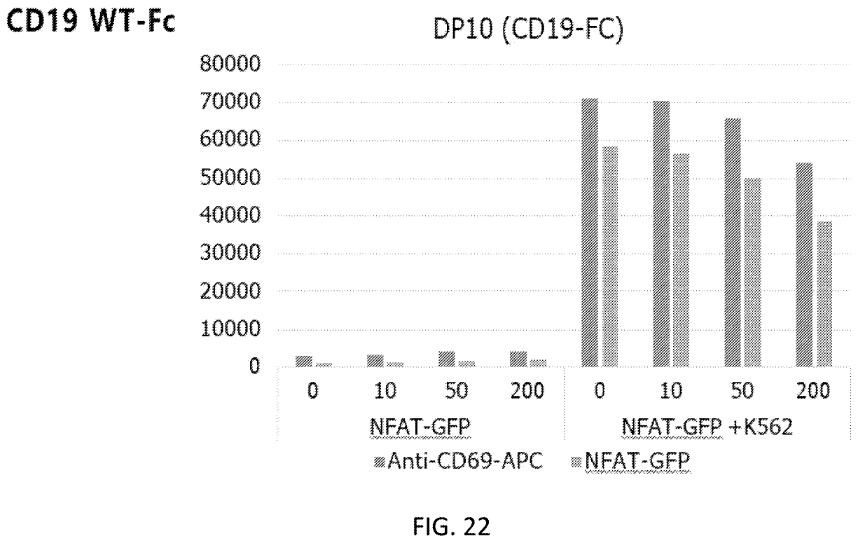

To test the activity of the variants, the Jurkat-K562 cell assay described above was used. FIG. 14 shows data indicating that the variants had improved IC50's as compared to the previous receptor traps. The data shown in FIGS. 15-17 show that the inhibition caused by the receptor traps is reversible. The data in FIGS. 18-22 show that the receptor traps minimally influence the Jurkat CAR-T cells in the absence of the activating K562 tumor cells.

### Example 3—Receptor Traps for HER2- and BCMA-Specific CARs

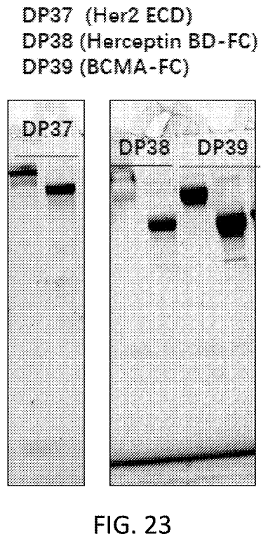

FIG. 23 illustrates gel analysis of receptor traps specific for Her2- and BCMA-specific CARs.

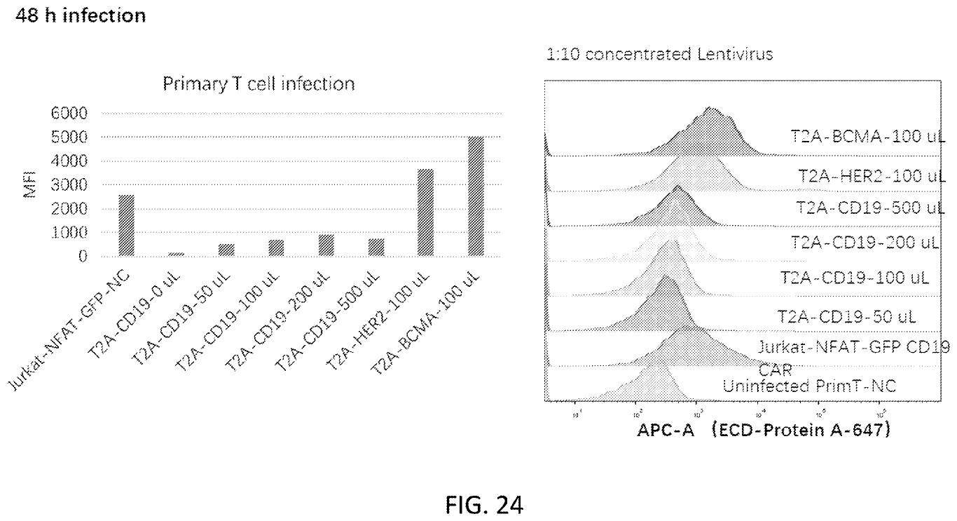

FIG. 24 shows expression of CARs, including Her2- and BCMA-specific CARs in primary T cells using retroviral vectors.

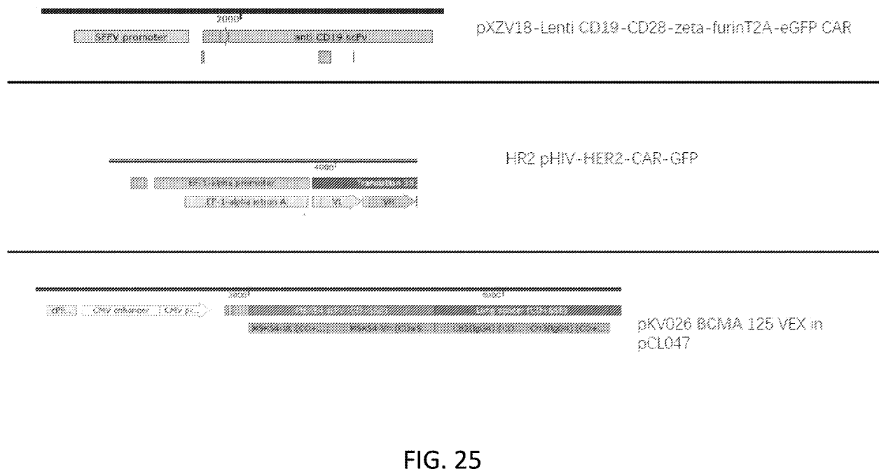

FIG. 25 shows schematics of the retroviral vectors used to express the CARs in the previous figure.

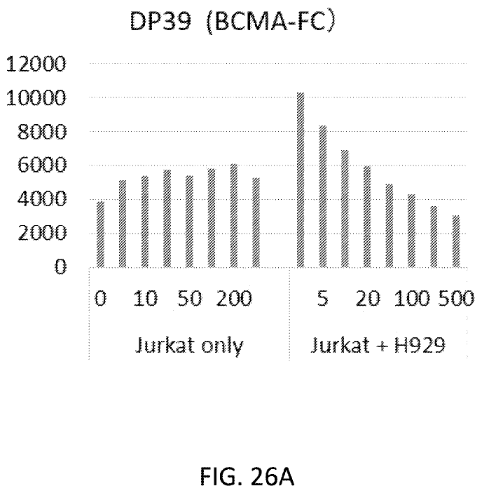

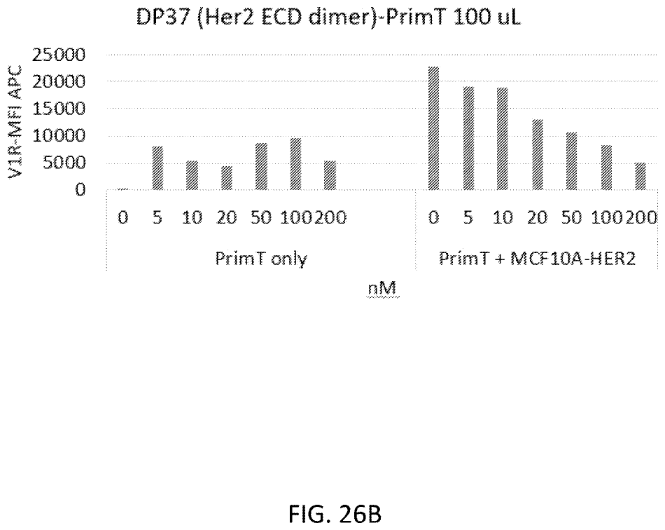

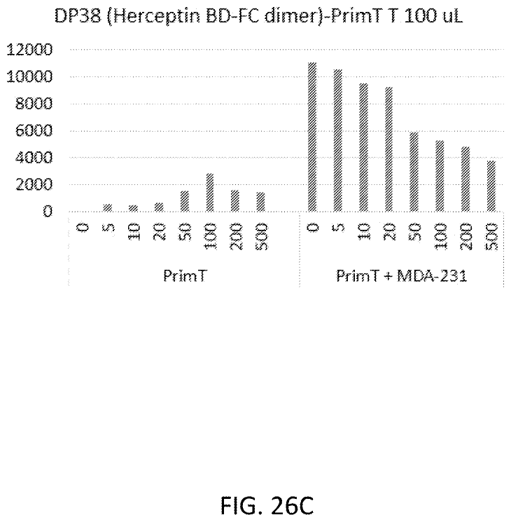

FIG. 26A shows the effects of an BCMA-specific receptor trap.

FIG. 26B shows the effects of a HER2-specific receptor trap.

FIG. 26C shows the effects of another HER-2-specific receptor trap.

### Example 4—Effects of CAR-Specific Receptor Traps (CAR-Trap) on CAR-T Cells

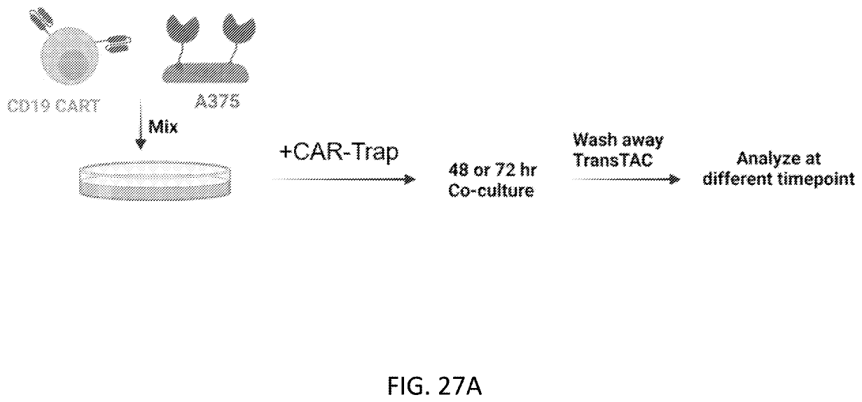

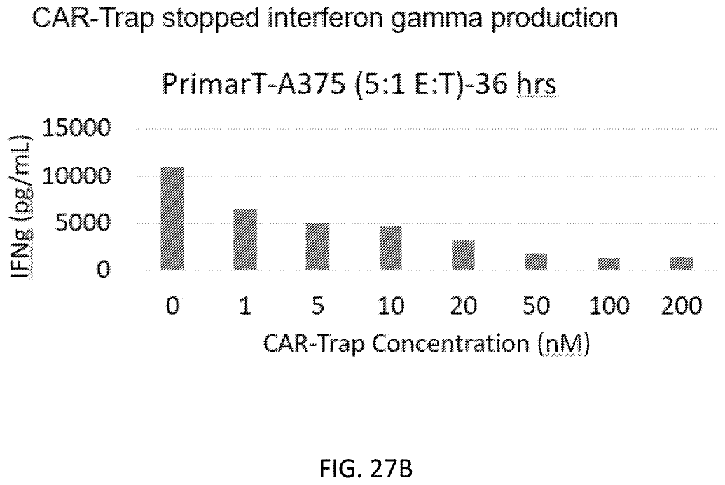

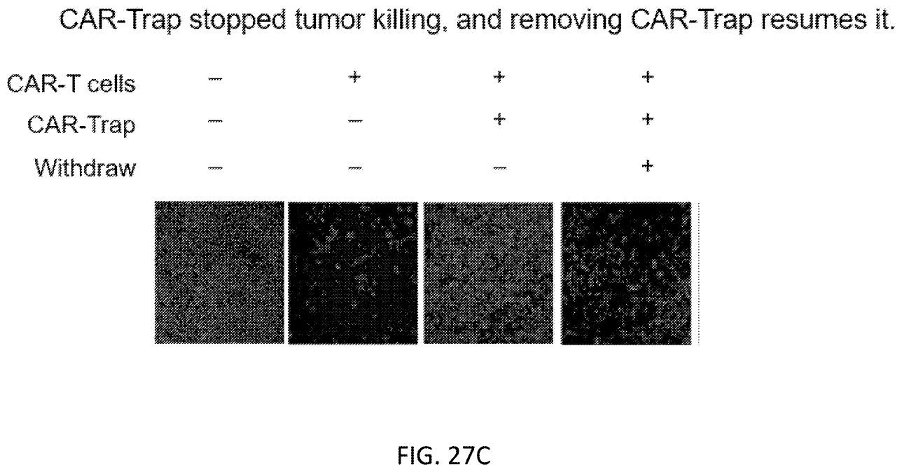

FIG. 27A shows a schematic diagram of this study.

FIG. 27B shows results indicating that a CAR-specific antigen trap interfered with interferon gamma production by a primary CAR-T cell.

FIG. 27C shows results indicating that a CAR-specific antigen trap interfered with tumor-cell killing of CAR-T cells. The data also show that the tumor-cell killing resumed when the antigen trap was removed.

### Example 5—Characterization of CD19 Ectodomain Variants for Inhibiting CAR-T Cell Activities

A CD19 CAR-Trap (wtCD19 CAR-Trap, FIG. 28A), was engineered by cloning and expressing the ectodomain of a wild-type CD19 (CD19-wt), consisting of amino acids 20-291, fused to the Fc portion of an IgG1 (CD19 wt-Fc) (FIG. 28A). To assess the inhibitory effect of this protein, we used a Jurkat/tumor co-culture assay. A CAR-Jurkat cell line expressing a nuclear factor of activated T cells (NFAT)-GFP activation marker was engineered and used as a CAR-T cell model to assess the inhibitory effect of the CD19 CAR-Trap), while a human immortalized myelogenous leukemia cell line K562, which overexpresses CD19, represented the cancer cells. An overnight co-incubation of these cell lines resulted in a 59-fold increase in the NFAT-GFP signals in CAR-Jurkats, indicating robust tumor-antigen-induced CAR-Jurkat activation. Increasing doses of CD19 wt-Fc added to the co-culture resulted in a dose-dependent decrease in Jurkat activation (FIG. 28B). However, the IC50 value was suboptimal (>200 nM), and only ˜30% of NFAT-GFP was downregulated at the maximum concentration tested.

Protein SDS-PAGE electrophoresis revealed high-order oligomers of the CD19 wt-Fc protein, indicating misfolding or formation of non-specific intermolecular interactions (FIG. 28C). Consistently, the expression yield of this protein was exceedingly low (FIG. 28D). This indicates that CD19-wt ectodomains may not be inherently stable.

CD19 mutants exhibiting high stability (19.1, C6.2, NT. 1, and CT.2; FIG. 28A) were expressed as fusions to IgG1 Fc (FIG. 28A). These mutants displayed as homogenous bands on an SDS-PAGE gel (FIG. 28C) and demonstrated significantly improved inhibitory potency, with IC50s ranging from 5-7 nM—an approximately 50-fold improvement compared to CD19 wt-Fc (FIG. 28B). The protein expression yields were increased by 79-208 fold, compared to CD19 wt-Fc (FIG. 28D).

CD19NT.1 was selected for further analysis based on the IC50s and protein expression yields observed (FIG. 28B, C). Structural analysis showed that mutations in CD19NT. 1 were positioned distantly from the interaction interface, suggesting these mutations predominantly contribute to the enhancement of CD19 ectodomain stability (FIG. 28E).

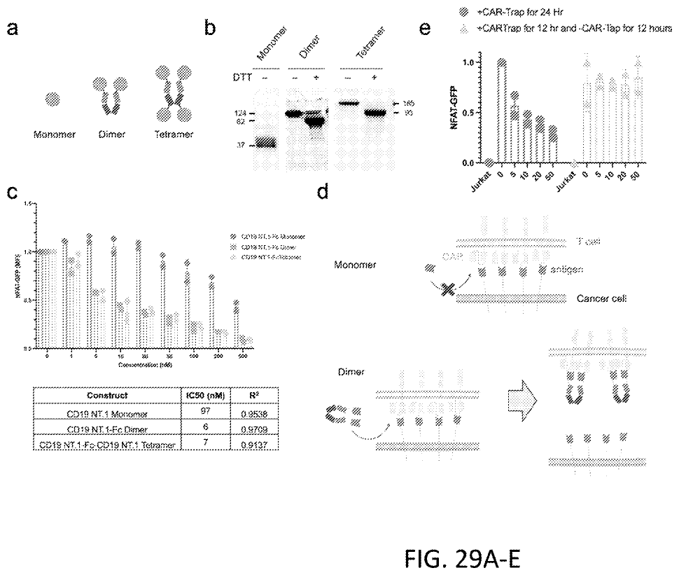

Additional experiments investigated multivalency in inhibiting CAR-Jurkat/tumor cell interactions. The multivalency of a molecule can enable it to bind multiple targets simultaneously, thereby increasing its overall binding strength, or ‘avidity’. Since a CAR-T/cancer cell interaction entails multiple copies of CD19s and CARs binding at the cell-cell interface, a multivalent CAR-Trap molecule can be more effective than a monovalent molecule to block this interaction. The inhibitory activities of monomeric, dimeric, and tetrameric CAR-Traps expressing the CD19NT. 1 domain either on its own, N-terminal to the IgG1 Fc, or N- and C-terminal to the IgG1, were assessed using the inhibition assay described above (FIG. 29A, B). The dimer and tetramer exhibited comparable IC50s, while the monomer showed an >80× lower potency, underscoring the significance of multivalency (FIG. 29C, D). These results show that enhancing the stability and avidity of a natural CD19 ectodomain can lead to a potent protein-based OFF-switch for regulating anti-CD19 CAR-Jurkat cells.

### Example 6—CAR Density Influences the T Cell Response to CAR-Traps and Tumor Cells

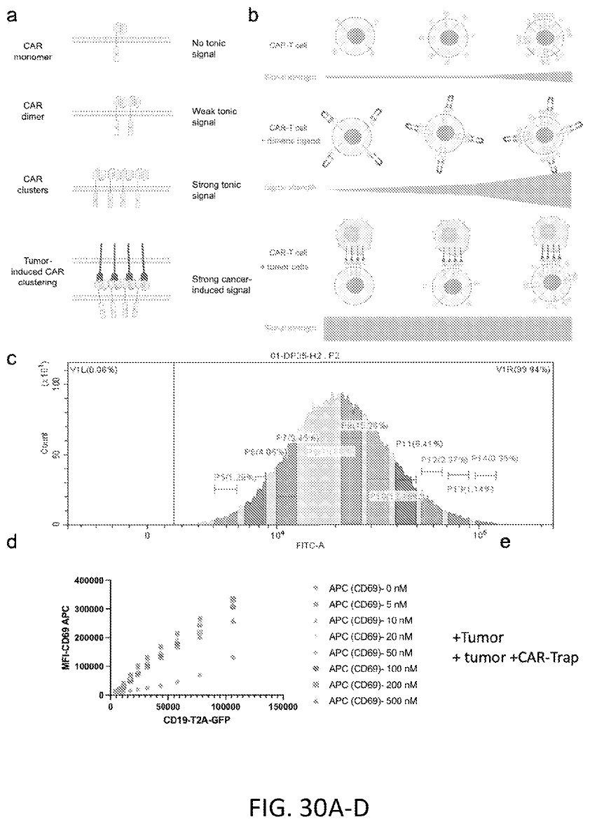

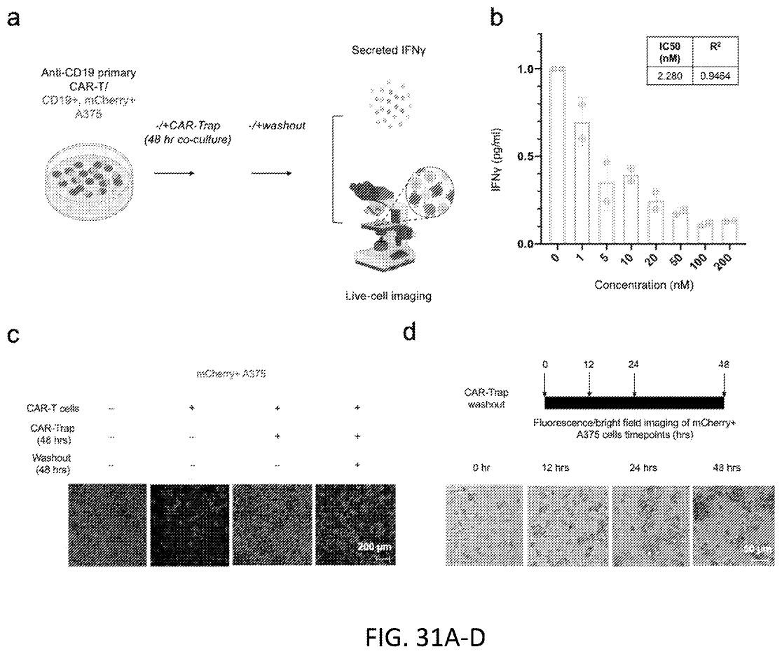

Recent findings indicate that the density of CAR molecules within the T cell membrane can influence CAR signaling and the overall efficacy of CAR-T therapy. To assess whether CAR density can impact the baseline, ligand-independent tonic signaling as well as the response to both CAR-Traps and tumor cells (FIG. 30A, B). Jurkat cells were infected with an EF1a-CAR construct incorporating a CD28 costimulatory domain and sorted into ten different expression groups (FIG. 30C). These groups were either untreated or exposed to varying concentrations of CAR-Traps, or tumor cells at a 1:1 effector: T cell ratio. Levels of CD69, an early T cell activation marker, were measured to assess T cell activation status under different treatment conditions.

As shown in FIG. 30D, there was a gradual increase in baseline CAR activation as the density of CAR molecules increased. The correlation between CD69 levels and CAR expression followed an exponential curve, indicating that more signaling-competent complexes formed as CAR expression increased.

CAR-Trap treatment can amplify CAR-T cell activities, by facilitating CAR dimerization (FIG. 30D). The relationship here appeared almost linear, indicating a direct correlation between CAR density and CAR-Trap induced activation. Contrarily, the response to tumor cells didn't uniformly increase across all CAR expression levels. Tumor-induced activation appeared to reach a saturation point as CAR expression increased, with no further activation observed in the highest expression groups.

To extrapolate these findings to primary CAR-T cells, we compared their expression levels with our ten CAR-Jurkat groups. We isolated primary human CD8+ T cells using established Ficoll separation protocols, generated CAR-T cells using lentiviruses, and compared their cell surface CAR expression to the CAR-Jurkat groups. The surface expression of CAR in primary T cells resembled that of group 1, suggesting that CAR-Traps would likely lead to minimal baseline activation and serve as effective inhibitors in these contexts.

To investigate behavior of clinically-produced CAR-T cells and their response to CAR-Traps, we quantified the density of cell surface CAR in our cell line system and compared it to numbers reported for clinical CAR-T cells. Clinical CAR-T cell products had similar CAR densities to the lowest expression population in our experimental setup, containing 1×10³-1×10⁵ CAR molecules per T cell. Thus, CAR-Traps can function as inhibitors in these scenarios, inducing minimal baseline activation.

These results show the significance of CAR densities in determining baseline tonic signaling, response to CAR-Traps, and response to tumor cells. Quantification and comparison of cell surface CAR in primary CAR-T cells and clinical CAR-T products indicates that CAR-Traps do not significantly trigger tonic signaling in these contexts, instead functioning predominantly as inhibitors.

### Example 7—CAR Density Influences the T Cell Response to CAR-Traps and Tumor Cells

To assess the reversibility of the inhibition activities described above, Anti-CD19 CAR-Jurkat cells and K562 cells were co-incubated for 12 hours with CAR-Trap. CAR-Trap was removed from the co-culture assay after 12 hours. The cells are subsequently incubated overnight with or without CAR-Trap, and NFAT-GFP was measured the following day. As shown in FIG. 29E, for all tested concentrations, the CAR-Jurkat cells resumed their activation without CAR-Trap, showing the reversibility of the CAR-Trap switch.

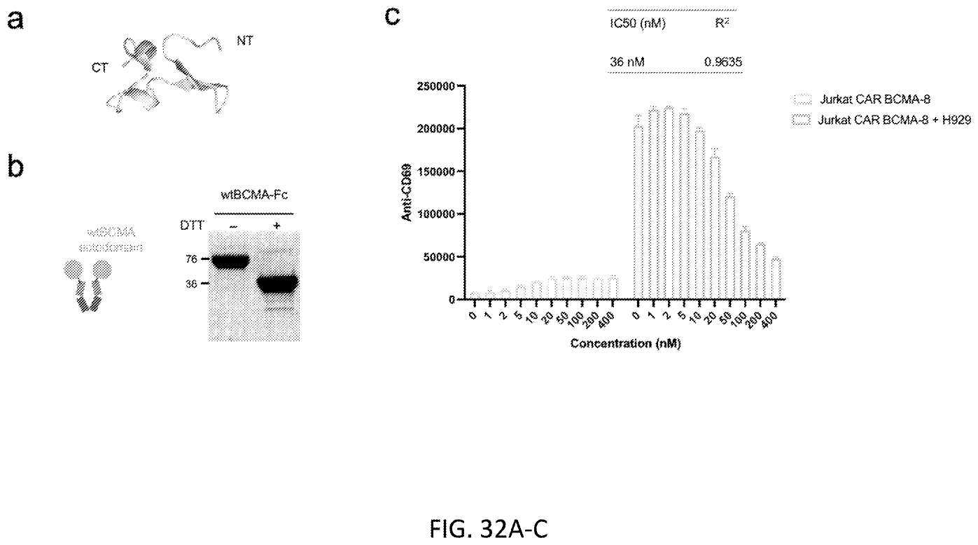

To determine if CD19 CAR-Trap could control the activities of primary human CAR-T cells, CD19+ A375 cells were used in wash out assays as depicted in FIG. 31A. In the absence of CAR-Trap, CD19+ A375 cells triggered the activation of primary human CAR-T cells and induced the release of interferon gamma (IFN-γ) (FIG. 31B). These CAR-T cells mediated antitumor effects. Fluorescence microscopy of mCherry-labeled A375 cells revealed CAR-T cell-mediated killing of A375 cells after 48 hours of co-culture (FIG. 31C). When co-incubated with CAR-Trap, a dose-dependent inhibition of IFN-γ release was observed, with an IC50 of 2 nM (FIG. 31B).

### Example 8—A BCMA-CAR Protein Switch

A BCMA CAR-Trap, was developed by replacing the CD19NT. 1 domain of CD19 NT.1-Fc Dimer with the ectodomain of BCMA (aa2 to 54) within the structure of the dimeric CAR-Trap molecule (FIG. 32A, B). This BCMAwt-Fc fusion protein expressed well and appeared as a homogeneous band on SDS-PAGE gel (FIG. 32A, B).

To assess the inhibitory effects of BCMAwt-Fc, CAR-Jurkat cells expressing a BCMA-CAR with a CD3zeta signaling motif, were incubated overnight with NCI-H929 cells, a BCMA-overexpressing human plasma cell line widely used for studying multiple myeloma. As shown in FIG. 32E, a 3-fold increase in the CD69 signals in CAR-Jurkats was observed after overnight incubation, indicating robust activation of CAR-Jurkat cells by the cancer cells. Addition of increasing doses of BCMAwt-Fc to the co-culture led to a dose-dependent decrease in Jurkat cell activation, with an IC50 of 36 nM (FIG. 32C). The BCMA CAR-Trap also inhibited CAR-Jurkat cell activation mediated by MM1.S cells, another B lymphoblast cell line.

### EQUIVALENTS

Those skilled in the art will recognize, or be able to ascertain, using no more than routine experimentation, numerous equivalents to the specific substances and procedures described herein. Such equivalents are considered to be within the scope of this invention.

What is claimed:

1. A receptor trap, comprising:
an ectodomain means from a tumor-specific antigen (TSA) or a tumor-associated antigen (TAA) that binds to a chimeric antigen receptor (CAR) on a CAR-T cell, and
a dimerization means;
wherein binding of the antigen trap to the CAR inhibits binding of the CAR to the TSA or TAA on a tumor cell and inhibits activation of the CAR-T cell.

2. The receptor trap of claim 1, wherein the dimerization means comprises an IgG antibody Fc domain fused to the ectodomain means.

3. The receptor trap of claim 2, wherein a linkage between the ectodomain means and the IgG antibody Fc domain comprises a glycine-rich linker.

4. The receptor trap of any one of claims 1-3, wherein the receptor trap comprises two or more ectodomain means.

5. A dimer of the receptor trap of any one of claims 1-4.

6. The receptor trap of any one of claims 1-5, wherein the ectodomain means comprises an ectodomain from CD19 or B-cell maturation antigen (BCMA).

7. The receptor trap of any one of claims 1-6, wherein the ectodomain means comprises a molecule having an amino acid sequence SEQ ID NO: 26 or 27, or an amino acid sequence 90, 91, 92, 93, 94, 95, 96, 97, 98 or 99% identical thereto.

8. The receptor trap of any one of claims 1-7, wherein the ectodomain means comprises a variant CD19 ectodomain with at least a 10-, 20-, 30-, 40-, or 50-fold lower IC50 than that of a wild-type CD19 ectodomain.

9. A method for treating side effects of CAR-T cell therapy or CAR-T cell exhaustion in a cancer patient, comprising administering the receptor trap of any one of claims 1-8 to the cancer patient.

10. The method of claim 9, wherein the cancer patient has received a CAR-T cell therapy for a cancer selected from the group consisting of B-cell acute lymphoblastic leukemia (ALL), B-cell non-Hodgkin lymphoma (NHL), follicular lymphoma, mantle cell lymphoma (MCL), and multiple myeloma.

11. The method of claim 9 or 10, wherein the side effects comprise cytokine release syndrome (CRS) or neurological toxicity.

12. The receptor trap of any one of claims 1-8 for use in treating side effects of CAR-T therapy or CAR-T cell exhaustion in a cancer patient.

13. A receptor trap, comprising:
a recombinant protein comprising a multivalent ectodomain or variant thereof from CD19 or B-cell maturation antigen (BCMA), and an IgG antibody Fc domain;
wherein binding of the multivalent ectodomain or variant thereof by a chimeric antigen receptor (CAR) on a CAR-T cell reversibly inhibits activation of the CAR-T cell.

14. The receptor trap of claim 13, wherein a linkage between the multivalent ectodomain or variant and the IgG antibody Fc domain comprises a glycine-rich linker.

15. The receptor trap of claim 13 or 14, wherein the ectodomain comprises SEQ ID NO: 26 or 27, or a sequence 90, 91, 92, 93, 94, 95, 96, 97, 98 or 99% identical thereto.

16. The receptor trap of any one of claims 13-15, wherein the ectodomain comprises a variant of a CD19 or BCMA ectodomain with at least a 10-, 20-, 30-, 40-, or 50-fold lower IC50 than that of a wild-type CD19 or BCMA ectodomain.

17. The receptor trap of any one of claims 13-16 comprising SEQ ID NO. 2, 4, 6, 8, 10, 12, 14, 16, 20, 22, 24, or an amino acid sequence 90, 91, 92, 93, 94, 95, 96, 97, 98 or 99% identical thereto.

18. A dimer of the receptor trap of any one of claims 13-17.

19. A nucleotide sequence encoding the receptor trap of any one of claim 1-8 or 13-18.

20. The nucleotide sequence of claim 19, comprising SEQ ID NO: 2, 4, 6, 8, 10, 12, 14, 16, 20, 22, 24 or a nucleotide sequence 90, 91, 92, 93, 94, 95, 96, 97, 98 or 99% identical thereto.

21. A pharmaceutical composition, comprising the receptor trap or the dimer of the receptor trap of any one of claim 1-8 or 13-18.
